(19) United States
(12) Patent Application Publication
(10) Pub. No.: US 2013/0273557 A1
(43) Pub. Date: Oct. 17, 2013

(54) COMPOUNDS AND METHODS OF MODULATING MITOCHONDRIAL BIOENERGETIC EFFICIENCY THROUGH AN INTERACTION WITH ATP SYNTHASE (COMPLEX V) AND ITS SUBUNITS

(71) Applicants: Valentin Gribkoff, Wallingford, CT (US); Steven Dworetzky, Pittsburgh, PA (US); Elizabeth Ann Jonas, Hamden, CT (US); Kambiz Nassirpour Alavian, New Haven, CT (US)

(72) Inventors: Valentin Gribkoff, Wallingford, CT (US); Steven Dworetzky, Pittsburgh, PA (US); Elizabeth Ann Jonas, Hamden, CT (US); Kambiz Nassirpour Alavian, New Haven, CT (US)

(21) Appl. No.: 13/699,304

(22) PCT Filed: May 26, 2011
(86) PCT No.: PCT/US11/38159

§ 371 Date: Jul. 1, 2013

Related U.S. Application Data
(60) Provisional application No. 61/348,698, filed on May 26, 2010.
(60) Provisional application No. 61/452,076, filed on Mar. 11, 2011.

(51) Int. Cl. G01N 33/94

(52) U.S. Cl. CPC G01N 33/94
USPC 435/7.1

(57) ABSTRACT

The present invention provides, in part, methods of identifying compounds that can bind to an ATP synthase complex, increase bioenergetic efficiency, decrease oxygen consumption or the rate thereof, increase oxygen utilization efficiency, increase cell survival or any combination thereof, and methods of using compounds and/or identified compounds to increase bioenergetic efficiency, increase oxygen utilization efficiency, decrease oxygen consumption, increase cell survival, or any combination thereof.

### CROSS-REFERENCE TO RELATED APPLICATIONS

This application claims priority to U.S. Provisional Application No. 61/348,698 entitled “Compounds and Methods of Modulating Mitochondrial ATP Synthase”, filed May 26, 2010, and U.S. Provisional Application No. 61/452,076, entitled “Compounds and Methods of Modulating Mitochondrial Bioenergetic Efficiency through an interaction with ATP Synthase (Complex V) and its Subunits”, filed Mar. 11, 2011, each of which is hereby incorporated by reference in its entirety.

### GOVERNMENT INTERESTS

This invention was made with government support under NS064967 and NS045876 awarded by NIH National Institute of Neurological Disorders and Stroke. The government has certain rights in the invention.

### PARTIES TO A JOINT RESEARCH AGREEMENT

Not Applicable

### INCORPORATION BY REFERENCE OF MATERIAL SUBMITTED ON A COMPACT DISC

Not Applicable

### BACKGROUND

1. Field of Invention

Not applicable

2. Description of Related Art

Not applicable

### BRIEF SUMMARY OF THE INVENTION

Embodiments of the present invention relate to novel compounds and methods of identifying or screening compounds suitable for the treatment of diseases where mitochondrial ATP synthase plays a significant role. Embodiments of the present invention also relate to specific compounds identified by such methods, wherein the compound interacts, binds or otherwise modulates mitochondrial ATP synthase.

### DESCRIPTION OF DRAWINGS

The file of this patent contains at least one photograph or drawing executed in color. Copies of this patent with color drawing(s) or photograph(s) will be provided by the Patent and Trademark Office upon request and payment of necessary fee.

For a fuller understanding of the nature and advantages of the present invention, reference should be had to the following detailed description taken in connection with the accompanying drawings, in which:

FIG. 1 shows dexpramipexole inhibited PSI-induced currents in brain-derived mitochondria.

FIG. 2 shows cyclosporine A (CSA) reduced peak conductance in dexpramipexole-sensitive PSI-mitochondria, and high calcium (Ca2+) induced dexpramipexole-sensitive currents in normal brain mitochondria, but dexpramipexole did not inhibit mitochondrial permeability transition recorded in rat liver mitochondria.

FIG. 3 shows dexpramipexole and CSA decreased conductance of submitochondrial vesicles (SMVs).

FIG. 4 shows the modulation of cellular bioenergetics by dexpramipexole.

FIG. 5 shows that dexpramipexole altered respiration parameters and ATP production in the C2C12 myoblast cell line.

FIG. 6 shows the modulation of complex V activity by dexpramipexole; Urea-treatment of SMVs alters enzymatic activity, pharmacology of membrane currents and radiolabeled dexpramipexole binding; and binding of radiolabeled dexpramipexole to individual heterologously-expressed subunits of complex V and competition by unlabeled dexpramipexole.

### DETAILED DESCRIPTION

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the methods, devices, and materials are now described. All publications mentioned herein are incorporated by reference in their entirety. Nothing herein is to be construed as an admission that the invention is not entitled to antedate such disclosure by virtue of prior invention.

Optical Isomers-Diastereomers-Geometric Isomers—Tautomers. Compounds described herein may contain an asymmetric center and may thus exist as enantiomers. Where the compounds according to the invention possess two or more asymmetric centers, they may additionally exist as diastereomers. The present invention includes all such possible stereoisomers as substantially pure resolved enantiomers, racemic mixtures thereof, as well as mixtures of diastereomers. The formulas are shown without a definitive stereochemistry at certain positions. The present invention includes all stereoisomers of such formulas and pharmaceutically acceptable salts thereof. Diastereoisomeric pairs of enantiomers may be separated by, for example, fractional crystallization from a suitable solvent, and the pair of enantiomers thus obtained may be separated into individual stereoisomers by conventional means, for example by the use of an optically active acid or base as a resolving agent or on a chiral HPLC column. Further, any enantiomer or diastereomer of a compound of the general formula may be obtained by stereospecific synthesis using optically pure starting materials or reagents of known configuration.

It must also be noted that as used herein and in the appended claims, the singular forms “a”, “an”, and “the” include plural reference unless the context clearly dictates otherwise. Thus, for example, reference to a “fibroblast” is a reference to one or more fibroblasts and equivalents thereof known to those skilled in the art, and so forth.

As used herein, the term “about” means plus or minus 10% of the numerical value of the number with which it is being used. Therefore, about 50% means in the range of 45%-55%.

“Administering” when used in conjunction with a therapeutic means to administer a therapeutic directly into or onto a target tissue or to administer a therapeutic to a patient whereby the therapeutic positively impacts the tissue to which it is targeted.

“Administering” a composition may be accomplished by oral administration, injection, infusion, absorption or by any method in combination with other known techniques.

The term “animal” as used herein includes, but is not limited to, humans and non-human vertebrates such as wild, domestic and farm animals.

The term “inhibiting” includes the administration of a compound of the present invention to prevent the onset of the symptoms, alleviating the symptoms, or eliminating the disease, condition or disorder. In some embodiments, inhibiting refers to at least about 10, 20, 30, 40, 50, 60, 70, 80, 90, 95, or 99% inhibition. In some embodiments, inhibiting refers to at least about 10-99, 20-99, 30-99, 40-99, 50-99, 60-99, 70-99, 80-99, or 90-99% inhibition With reference to inhibiting the progression of a disease, the inhibition need not be 100%. That is in the absence of a compound the disease would progress more rapidly, then as compared to in the presence of a compound, then the compound is said to inhibit the progression of the disease. In some embodiments, a compound can inhibit the progression of a disease completely or incompletely. An incomplete inhibition means that the disease may progress but at a slower rate than in the absence of the compound. Progression of a disease can be measured by known criteria. For example, for a neurodegenerative disease, progression can be measured/determined by measuring cognitive ability, motor activity, and the like.

By “pharmaceutically acceptable”, it is meant the carrier, diluent or excipient must be compatible with the other ingredients of the formulation and not deleterious to the recipient thereof.

The term “target,” as used herein, refers to the material for which either deactivation, rupture, disruption or destruction or preservation, maintenance, restoration or improvement of function or state is desired. For example, diseased cells, pathogens, or infectious material may be considered undesirable material in a diseased subject and may be a target for therapy.

The term “improves” is used to convey that the present invention changes either the appearance, form, characteristics and/or physical attributes of the tissue to which it is being provided, applied or administered. “Improves” may also refer to the overall physical state of an individual to whom an active agent has been administered. For example, the overall physical state of an individual may “improve” if one or more symptoms of a neurodegenerative disorder are alleviated by administration of an active agent.

As used herein, the term “therapeutic” means an agent utilized to treat, combat, ameliorate or prevent an unwanted condition or disease of a patient.

The terms “therapeutically effective amount” or “therapeutic dose” as used herein are interchangeable and may refer to the amount of an active agent or pharmaceutical compound or composition that elicits a biological or medicinal response in a tissue, system, animal, individual or human that is being sought by a researcher, veterinarian, medical doctor or other clinician. A biological or medicinal response may include, for example, one or more of the following: (1) preventing a disease, condition or disorder in an individual that may be predisposed to the disease, condition or disorder but does not yet experience or display pathology or symptoms of the disease, condition or disorder, (2) inhibiting a disease, condition or disorder in an individual that is experiencing or displaying the pathology or symptoms of the disease, condition or disorder or arresting further development of the pathology and/or symptoms of the disease, condition or disorder, and (3) ameliorating a disease, condition or disorder in an individual that is experiencing or exhibiting the pathology or symptoms of the disease, condition or disorder or reversing the pathology and/or symptoms experienced or exhibited by the individual.

As used herein, the term “neuroprotectant” refers to any agent that may prevent, ameliorate or slow the progression of neuronal degeneration and/or neuronal cell death, or protects neurons from the toxic actions of another agent. A “neuroprotectant” can also refer to any agent that may prevent, ameliorate or slow the progression of a central nervous cell degeneration and/or central nervous cell death, or protects a central nervous cell from the toxic actions of another agent.

As used herein, the term “central nervous system cell” refers to a cell that is part of the central nervous system. Examples include, but are not limited to neurons and glia cells. Examples of neurons and glia cells include, but are not limited to, oligodendrocytes, sensory neurons, motor neurons, Schwann cells, astrocytes, and the like.

For the purposes of this disclosure, a “salt” is any acid addition salt, preferably a pharmaceutically acceptable acid addition salt, including but not limited to, halogenic acid salts such as hydrobromic, hydrochloric, hydrofluoric and hydroiodic acid salt; an inorganic acid salt such as, for example, nitric, perchloric, sulfuric and phosphoric acid salt; an organic acid salt such as, for example, sulfonic acid salts (methanesulfonic, trifluoromethan sulfonic, ethanesulfonic, benzenesulfonic or p-toluenesulfonic), acetic, malic, fumaric, succinic, citric, benzoic, gluconic, lactic, mandelic, mucic, pamoic, pantothenic, oxalic and maleic acid salts; and an amino acid salt such as aspartic or glutamic acid salt. The acid addition salt may be a mono- or di-acid addition salt, such as a di-hydrohalogenic, di-sulfuric, di-phosphoric or di-organic acid salt. In all cases, the acid addition salt is used as an achiral reagent which is not selected on the basis of any expected or known preference for interaction with or precipitation of a specific optical isomer of the products of this disclosure.

“Pharmaceutically acceptable salt” is meant to indicate those salts which are, within the scope of sound medical judgment, suitable for use in contact with the tissues of a patient without undue toxicity, irritation, allergic response and the like, and are commensurate with a reasonable benefit/risk ratio. Pharmaceutically acceptable salts are well known in the art. For example, Berge et al. (1977) J. Pharm. Sciences, Vol 6. 1-19, describes pharmaceutically acceptable salts in detail.

The term “treating” may be taken to mean prophylaxis of a specific disorder, disease or condition, alleviation of the symptoms associated with a specific disorder, disease or condition and/or prevention of the symptoms associated with a specific disorder, disease or condition. In some embodiments, the term refers to slowing the progression of the disorder, disease or condition or alleviating the symptoms associated with the specific disorder, disease or condition. In some embodiments, the term refers to slowing the progression of the disorder, disease or condition. In some embodiments, the term refers to alleviating the symptoms associated with the specific disorder, disease or condition. In some embodiments, the term refers to restoring function which was impaired or lost due to a specific disorder, disease or condition.

The term “patient” generally refers to any living organism to which compounds described herein are administered and may include, but is not limited to, any non-human mammal, primate or human. Such “patients” may or my not be exhibiting the signs, symptoms or pathology of the particular diseased state.

As used herein, KNS-760704 is dexpramipexole (((6R)-2-amino-4,5,6,7-tetrahydro-6-(propylamino)benzothiazole)), which is a synthetic aminobenzothiazole derivative.

The (6S) enantiomer of KNS-76704, commonly known as pramipexole and commercially available under the Mirapex® name, is a potent dopamine agonist, which mimics the effects of the neurotransmitter dopamine. Pramipexole has also been shown to have both neuroprotective and dopaminergic activities, presumably through inhibition of lipid peroxidation, normalization of mitochondrial metabolism and/or detoxification of oxygen radicals. Therefore, pramipexole may have utility as an inhibitor of the cell death cascades and loss of cell viability observed in neurodegenerative diseases such as Parkinson's disease. Additionally, oxidative stress caused by an increase in oxygen and other free radicals has been associated with the fatal neurodegenerative disorder amyotrophic lateral sclerosis (ALS), a progressive neurodegenerative disorder involving the motor neurons of the cortex, brain stem, and spinal cord.

The structures of pramipexole and KNS-76704 (dexpramipexole are depicted below):

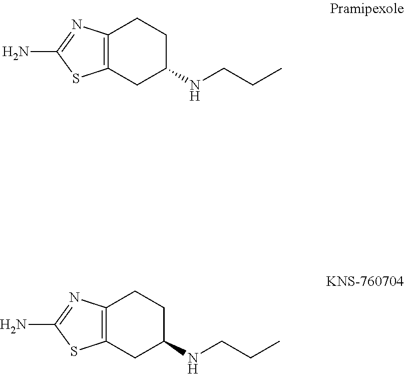

While not wishing to be bound by theory, dexpramipexole has been demonstrated to have effects in normal and stressed mitochondria that are consistent with the interpretation that the compound increases bioenergetic efficiency by suppressing aberrant large-conductance currents in mitochondrial membranes. Such currents provide a source of uncoupling between oxygen and metabolic substrate use and oxidative phosphorylation, and may represent a leak which reduces the proton-motive force driving ATP production. Dexpramipexole has been shown to be neuroprotective in multiple in vitro and in vivo assays. While its mechanism of action remains to be fully characterized, recent studies have localized the target responsible for its neuroprotective properties to the level of the mitochondrion. Dexpramipexole produces a concentration-dependent inhibition of large-conductance ion channel activity produced in isolated rat brain mitochondria associated with neuronal cell death. Consistent with this pharmacology, dexpramipexole also produced significant concentration-dependent in vitro cytoprotection in human neuroblastoma SH-SY5Y cells exposed to the mitochondrial toxin MPP⁺ or the proteasome inhibitor PSI. An independent laboratory has shown that dexpramipexole significantly increased in vivo survival in the G93A-SOD1 mutant mouse model of ALS.

Proteasomal dysfunction has been implicated in a variety of neurodegenerative and aging-related disorders, including ALS, Parkinson's disease (PD), and Alzheimer's disease (AD). Decreased proteasome function results in abnormal accumulation of misfolded proteins and ultimately leads to cell death, possibly by inducing mitochondrial dysfunction. The proteasome inhibitor Z-Ile-Glu(OtBu)-Ala-Leu-H(PSI) is a synthetic peptide that reversibly inhibits the chymotrypsin-like activity of the proteasome and has been used in vivo to model the effects of PD and in vitro to elucidate potential neurotoxic mechanisms involved in these disorders.

Mitochondria derived ex vivo from subcortical brain regions of rats treated with PSI (but not from subcortical regions from control rats or from cortical regions in PSI-treated rats) exhibit unusual large-conductance ion channel activity similar to mitochondrial transition pore activity of the inner mitochondrial membrane. In some embodiments, dexpramipexole, when applied to these mitochondria under whole-cell mitochondrial voltage clamp, produces a concentration-dependent, potent, effective and reversible suppression of this conductance with little effect on lower-amplitude conductances seen in control mitochondria. Accordingly, in some embodiments, the methods described herein can be used to identify a compound that inhibits PSI's effect(s) on a cell. In some embodiments, a method is provided that identifies a compound that can suppress the effect of PSI. In some embodiments, the method comprises contacting a cell, mitochondria, or sub-mitochondrial vesicle with PSI in the absence or presence of a test compound. As described herein and is applicable to all cells, mitochondria, or sub-mitochondrial vesicles, the cells, mitochondria, or sub-mitochondrial vesicles can be isolated. The cells, mitochondria, or sub-mitochondrial vesicles can also be part of a subject or organism. The effect of the test compound is measured and if the test compound suppresses the effects of PSI, the compound is said to be able to suppress the effect of PSI. In some embodiments, the test compound is compared to a positive control's ability to suppress the effect of PSI. In some embodiments, the positive control is dexpramipexole.

As described herein, a method of identifying a compound can comprise contacting a test compound with an ATP synthase complex. In addition, to the ATP synthase complex described herein, in some embodiments, an ‘ATP synthasome’ can be used as the ATP synthase complex. An ATP synthasome, in some embodiments, is a preparation that isolates specific and critical components of the bioenergetic pathway of the mitochondrion, ATP synthase (complex V) and associated proteins inserted into a membrane and comprising a submitochondrial particle, and in which application of voltage through a patch clamp electrode can produce a large-conductance leak current, similar to the current described above in neuronal mitochondria derived from PSI-treated rats. In some embodiments, the ATP synthasome leak current can be inhibited by a compound identified herein. In some embodiments, the compound is dexpramipexole. In some embodiments, the compound has an EC₅₀ of about 100-120, 110-120, nM and any number in the range including the endpoints. The concentration-response relationships for current inhibition by dexpramipexole or a compound identified by a method described herein in both mitochondria and synthasomes can be very shallow (Hill slopes<<1).

Accordingly, in some embodiments, dexpramipexole or a compound identified by a method described herein may work by decreasing an insult-dependent or stress-augmented proton (H⁺) leak conductance that shunts the proton-motive force necessary to couple the electron transport chain to ATP production, and that components of the mitochondrial transition pore are localized in the complex producing the leak conductance. Attenuating, but more likely entirely inhibiting this leak conductance should improve mitochondrial bioenergetics by increasing the efficiency of ATP production. In some embodiments, such a mechanism would enable neurons to more effectively resist environmental stresses, including toxicity from abnormal protein aggregation and reactive oxygen species, which are associated with the development of many neurodegenerative diseases. Accordingly, in some embodiments, a compound identified by a method described herein can be used to increase mitochondrial bioenergetics. In some embodiments, the increase or improvement of mitochondrial bioenergetics comprises an increase in the efficiency of ATP production. In some embodiments, a compound identified using a method described herein can be used to inhibit leak conductance. In some embodiments, the method comprises contacting a cell or a subject with a compound identified by a method described herein, wherein the compound inhibits leak conductance.

The examples described herein using ¹⁴C-dexpramipexole, have shown that removal of a critical component of ATP synthase in synthasome preparations, the F1 ‘head’ of the synthase complex, greatly reduced (by as much as 80%) the binding of radiolabeled dexpramipexole to the synthasome. Similarly, while removal of the F1 head did not diminish the inhibition of ‘leak’ currents by ATP (indicating that leak inhibition by ATP occurs elsewhere in complex V), it abolished the inhibition by dexpramipexole. Accordingly, in some embodiments, dexpramipexole binds to a site in the ATP synthase complex, inhibiting a leak conductance in a cooperative manner. Therefore, additional compounds can be identified by identifying compounds that bind to the F1 head. In some embodiments, a compound that binds to the F1 head and can competitively inhibit the binding of dexpramipexole is identified as a compound that can increase mitochondrial bioenergetic efficiency. In some embodiments, the method comprises contacting a F1 head with a test compound and determining whether the test compound binds to the F1 head. In some embodiments, the method comprises contacting a F1 head with dexpramipexole (labeled or unlabeled) and a test compound either simultaneously or sequentially in any order and determining whether the test compound can bind to the F1 head or can inhibit the binding of dexpramipexole to the F1 head. In some embodiments, if the test compound can bind to the F1 head and can inhibit the binding of dexpramipexole to the F1 head, the compound is identified as a compound that can increase bioenergetic efficiency or can inhibit leak conductance. In some embodiments, an ATP synthase complex, such as, but not limited to, an ATP synthasome comprises the F1 head. In some embodiments, the ATP synthase complex is an ATP synthase complex as described herein.

In some embodiments, a high-throughput respirometry system can be used to show that, for example, dexpramipexole can reduce oxygen consumption by mitochondria without forcing cells to ‘switch’ to glycolysis to obtain needed ATP. This high-throughput system can also be used to identify compounds other than dexpramipexole that can reduce oxygen consumption without an increase in glycolysis to increase ATP. ATP levels in the presence of a compound, such as dexpramipexole, can remain constant or are even increased over a wide range of concentrations, direct evidence that the compound can improve bioenergetic efficiency even in non-stressed cells. These assays in conjunction with the binding to an ATP synthase complex can be used to identify compounds as described herein.

In some embodiments, dexpramipexole, a test compound, or a compound identified with a method described herein interacts (e.g. binds) with specific subunits of an ATP synthase complex. For example, nine specific subunits of ATP synthase can be heterologously expressed in a cell, such as, but not limited to, 293T cells, including the proteins comprising the alpha (α), beta (β), b, c, delta (δ), d, epsilon (ε), gamma (γ), and OSCP (oligomycin-sensitivity conferring protein). The human ORF constructs for these subunits can be tagged with Myc and DDK (Flag) tags, which can be obtained from Origene Technologies (Rockville, Md.). The cells can be transfected with the above constructs, using any transfection method, including a lipid mediated (e.g. LIPOFECTAMINE) or a calcium phosphate method. In some embodiments, on day 1, 2, or 3 post-transfection, the cells can be lysed and the fusion proteins were bound to an EZview™ Red ANTI-FLAG® M2 Affinity Gel, using a standard protocol. The proteins can be eluted from a portion of the samples and presence of the proteins on the beads can be verified by immunoblot analysis, using commercially available mouse anti-Myc antibodies. The protein-bound beads can then be incubated with a test compound that is labeled or unlabelled or a positive control such as dexpramipexole to determine whether the test compound can bind to the ATP synthase complex or a portion thereof. Any method can be used to detect the interaction. For example, if the compound is labeled (e.g. ¹⁴C-labeling) the amount of ¹⁴C-labeled compound bound to the beads can be measured to determine if the compound binds to the ATP synthase complex or a portion thereof, such as a subunit of the ATP synthase complex. The same type of method can be used to determine if a compound binds to the F1 head. Examples of methods are also described in the example section. The test compound can be compared to a positive control, such as dexpramipexole, or the ability for a test compound to bind to the ATP synthase complex can be measured by determining whether the test compound can inhibit the binding of the positive control to the ATP synthase complex. In some embodiments, a ¹⁴C-labeled dexpramipexole is used as a positive control (e.g. binding ligand).

In some embodiments, a method can be designed to determine whether the binding of ¹⁴C-dexpramipexole is specific and competitive. In some embodiments, the protein-bound beads can be co-incubated in ¹⁴C-dexpramipexole (e.g. 1-200 nM) and non-radiolabeled dexpramipexole at a concentration of, for example, 100 μM, a concentration, which can be, ˜500× the concentration of the radiolabeled compound. In some embodiments, there can be no effect on the untransfected control level of radioactivity, but the levels bound to both the b subunit and the OSCP subunit can be reduced to levels insignificantly different from the control levels. In some embodiments, dexpramipexole binds to one or more sites in the b and OSCP subunits of the F1F_{O} ATP synthase (complex V). In some embodiments, the b and OSCP subunits are closely associated, and form the ‘stator’ stalk believed to have a role in stabilizing F1 during rotation. In some embodiments, dexpramipexole binds to one or more sites in the F1F_{O} ATP synthase ‘stator’ stalk. In some embodiments, the interaction with this/these sites inhibits stress- or dysfunction-induced metabolic leak conductance in mammalian mitochondria, including dysfunctional mitochondria in humans. In some embodiments, a test compound is also used to determine whether the binding is specific and competitive. The binding method can be done in the presence or absence of a positive control (e.g. dexpramipexole).

In some embodiments, the inhibition of mitochondrial leak conductance increases bioenergetic efficiency. In some embodiments, multi-well and high-resolution respirometry systems are used with both whole cells in culture and isolated mitochondria from neurons to show the increase in bioenergetic efficiency. For example, dexpramipexole or a compound identified using a method described herein can be shown to decrease the basal consumption of oxygen (O₂) without, in whole cells, producing a compensatory switch in cell energy production to glycolysis. Accordingly, in some embodiments, the methods described herein can be used to identify a compound that decreases the basal consumption of oxygen (O₂) without, in whole cells, producing a compensatory switch in cell energy production to glycolysis. In some embodiments, this can be seen as shown in examples described herein, which show that in the presence of dexpramipexole, oxygen consumption rate (OCR) was decreased modestly with no accompanying change in ECAR (extracellular acidification rate), a measure of the production of lactic acid by glycolysis. In some embodiments, this indicates that the decrease in O2 consumption was not a result of toxicity or mitochondrial dysfunction, since it has been demonstrated that dexpramipexole over this concentration range either maintains or even increases cellular ATP levels, which indicates that the decrease in O₂ consumption in dexpramipexole reflects an increase in mitochondrial bioenergetic efficiency. In some embodiments, in cells incubated in the proteasome inhibitor PSI, used in the some experiments to induce leak conductances in subcortical mitochondria (conductances that were potently and effectively attenuated by dexpramipexole), oxygen consumption can be decreased significantly, in this case reflecting mitochondrial dysfunction, since ECAR is significantly increased. Accordingly, a compound that decreases oxygen consumption can be identified in the presence of PSI or in some other embodiments, in the absence of PSI.

In some embodiments, when a cell is simultaneously incubated with dexpramipexole, a compound identified as described herein, and/or PSI, O₂ consumption can be decreased, without a concomitant increase in ECAR. Therefore, in some embodiments, a method of identifying a compound that decreases oxygen consumption comprises contacting a cell with a test compound in the presence or absence of PSI and measuring oxygen consumption. In some embodiments, the test compound is compared to a positive control, such as dexpramipexole, wherein when the test compound decreases oxygen consumption the same or greater than the positive control the compound is said to be a compound that decreases oxygen consumption or the rate thereof. In some embodiments, when the rate of oxygen consumption or the amount of oxygen consumed for a test compound is done in the presence of PSI, and the test compound counters the effects of PSI-induced mitochondrial dysfunction the compound is be said to increase mitochondrial bioenergetic efficiency.

Therefore, in some embodiments, the present invention provides a method of identifying a compound that increases mitochondrial bioenergetic efficiency. In some embodiments, the method comprises contacting a test compound with a cell and measuring mitochondrial bioenergetic efficiency. In some embodiments, measuring mitochondrial bioenergetic efficiency comprises measuring oxygen consumption or the rate thereof. If the rate of oxygen consumption or the amount of oxygen consumed is decreased without a concomitant increase in glycolysis (e.g. ECAR) the compound is said to be a compound that increases mitochondrial bioenergetic efficiency. In some embodiments, the cell is also contacted with PSI, which does not increase mitochondrial bioenergetic efficiency. If the test compound can inhibit the effects of PSI, the compound is said to increase mitochondrial bioenergetic efficiency. In some embodiments, the test compound is compared to a positive control can increase mitochondrial bioenergetic efficiency. An example of a positive control is dexpramipexole. In some embodiments, a compound identified as described herein can increase maximal respiratory capacity, as well as spare respiratory capacity, a phenomenon that is particularly visible and pronounced when succinate is used as the electron donor via complex 2 of the electron transport system in the presence of the complex 1 inhibitor rotenone.

Accordingly, in some embodiments, dexpramipexole or a compound identified using the methods described herein increases cell survival. In some embodiments, the cell survival is increased in a model of PSI-induced proteasome dysfunction where another compound such as riluzole does not. In some embodiments, the compound, such as dexpramipexole or a compound identified using a method described herein, can exert a direct cytoprotective effect (e.g., not glial cell mediated) on neuronal cells challenged with proteasome inhibition under diverse treatment paradigms, and suggests that inhibition of mitochondrial ‘leak’ conductances likely mediate this protective effect. Thus, in some embodiments, a compound such as dexpramipexole or another identified by a method described herein or by specific interaction with one or more sites in the F1F_{O} ATP synthase can inhibit aberrant metabolic leak conductances in mitochondria, increasing bioenergetic efficiency in these mitochondria and acting to protect cells from a variety of toxins and disease conditions. In some embodiments, the method comprises contacting a cell or administering to a subject a therapeutically effective amount of a composition comprising a compound identified using a method described herein, wherein the compound increases cell survival. In some embodiments, the composition does not comprise dexpramipexole. In some embodiments, the method of increasing cell survival is combined with any method of identifying a compound as described herein.

These effects and the data described herein can be used in methods to identify other compounds that can increase cell survival, increase oxygen utilization efficiency, decrease the rate of oxygen consumption, and at least maintain ATP synthesis using the methods described herein (e.g. no increase in glycolysis). Although the methods described herein may recite a particular readout, each of the methods described herein can also be used to identify a compound that increases cell survival. In some embodiments, the method comprises measuring cell survival in the absence or presence of a compound or as compared to a positive or negative control. With respect to cell survival, a positive control is a compound that increases cell survival as compared to the cell survival in the absence of the positive control. The cell survival can be measured in the presence or absence of a cellular stress, such as, but not limited to, those described herein. With respect to cell survival, a negative control is a compound that has no effect on cell survival.

Some embodiments of the present invention relate to novel compounds and methods of identifying or screening compounds suitable for the treatment of diseases where mitochondrial ATP synthase plays a significant role. Embodiments of the present invention also relates to specific compounds identified by such methods which interact or modulate mitochondrial ATP synthase. In certain embodiments, the compounds inhibit mitochondrial ATP synthase activity. In each of the embodiments discussed herein, ATP synthase complex may be interchanged with ATP synthase or a portion thereof. That is, the term “ATP synthase complex” can refer to the complete ATP synthase complex or a portion of the complex. In some embodiments, a portion of the ATP synthase complex is the F1 head. In some embodiments, a portion of the ATP synthase complex refers to at least one the subunits, which include, for example, alpha (α), beta (β), b, c, delta (δ), d, epsilon (ε), gamma (γ), and OSCP (oligomycin-sensitivity conferring protein). In some embodiments, a portion of the ATP synthase complex refers to the F1 head, the b subunit, the OSCP subunit, or any combination thereof. The ATP synthase complex can also be an ATP synthasome, which is described herein.

In some embodiments, the present invention provides compositions comprising a crystal of a mitochondrial ATP synthase complex, wherein the complex includes the F1 ‘head’ of the mitochondrial ATP synthase complex. In certain embodiments, the ATP synthase complex includes its nine (9) subunits: the alpha (α), beta (β), b, c, delta (δ), d, epsilon (ε), gamma (γ), and OSCP (oligomycin-sensitivity conferring protein). In certain embodiments, the ATP synthase complex includes the b subunit and the OSCP subunit. In certain embodiments, the ATP synthase complex includes the b subunit. In certain embodiments, the ATP synthase complex includes the OSCP subunit.

In some embodiments, the ATP synthase complex is a mitochondrial ATP synthase complex. In some embodiment, the mitochondrial ATP synthase is an isolated mitochondrial ATP synthase complex. As used herein, the term “isolated mitochondrial ATP synthase complex” refers to an ATP synthase complex that has been isolated (e.g. purified) away from a mitochondria. The isolated ATP synthase complex can be functional or non-functional. Even if the ATP synthase complex is non-functional, in some embodiments, the ATP synthase complex retains its three-dimensional structure as if it were present in a mitochondria. In some embodiments, the ATP synthase complex is denatured. In some embodiments, the ATP synthase complex is not denatured.

In some embodiments of the present invention, assays for screening compounds that modulate the activity of mitochondrial ATP synthase are provided. In certain embodiments, compounds are identified as inhibitors of mitochondrial ATP synthase via comparison to, e.g., dexpramipexole.

These assays may be cell-based, consisting of establishing aberrant bioenergetic respiratory profiles following induction of dysfunction and testing the ability of compounds to re-establish, induce, or enhance bioenergetic efficiency in these cells, as indicated by, for example, recording the oxygen consumption rate (OCR) and extracellular acidification rate (ECAR) in response to additions of various substrates and under various experimental conditions. Substrates include but are not limited to succinate, glutamate, pyruvate, maleate and glucose. Experimental conditions include but are not limited to addition of adenosine di-phosphate (ADP) and the uncoupling compound FCCP, as well as the complex V inhibitor oligomycin. Cells useful in such assays may include clonal cell lines, derived from human and animal sources, as well as embryonic neurons grown in culture and other cells from animal sources. Such techniques of respiratory profiling can also be used as an assay employing isolated mitochondria, which may be neuronal in origin, derived acutely from experimental animals including transgenic models of disease, or they may be derived from other organ systems, such as, but not limited to, mitochondria from liver cells or fibroblasts. In special circumstances, peripheral cells can be harvested from patients; these may be used as acutely cultured cells, or mitochondria may be derived for assays.

These assays may also be based on the binding affinity of radiolabeled dexpramipexole to either native complex V in isolated mitochondrial membranes, or may utilize the heterologous expression of the b and OSCP subunits bound to beads as an assay. In such embodiments test compounds would be used to displace radiolabeled dexpramipexole; compounds would be ranked based on the IC₅₀ and K_{D} of such interactions.

In some embodiments, the present invention provides methods of identifying a compound that increases oxygen utilization efficiency. In some embodiments, the method comprises contacting a compound that binds a mitochondrial ATP synthase complex with a mitochondria or a sub-mitochondrial vesicle; and measuring oxygen utilization efficiency, wherein when the measured oxygen utilization efficiency in the mitochondria or a sub-mitochondrial vesicle increases in the presence of the compound that binds the mitochondrial ATP synthase complex indicates that the compound is a compound that increases oxygen utilization efficiency. The increase in oxygen utilization efficiency can be determined using any method. In some embodiments, the oxygen utilization efficiency is determined by comparing the oxygen consumption rate (OCR) in the presence and absence of the compound. A compound that decreases the amount of oxygen utilized by the cell while maintaining the cell's viability is a compound that increases the oxygen utilization efficiency. The oxygen utilization efficiency refers to the amount of oxygen that a cell requires for growth and/or survival. Therefore, if a cell requires more oxygen in the presence of a compound the oxygen utilization efficiency is said to decrease. If a cell requires less oxygen in the presence of a compound, the oxygen utilization efficiency is said to increase. The oxygen utilization efficiency can be determined by measuring and/or comparing the oxygen consumption rate as described herein and by other methods known.

In some embodiments, the compounds ability to have an effect on oxygen utilization efficiency is determined by comparing the compound to a positive or a negative control. A positive control compound is a compound that increases oxygen utilization efficiency. A negative control compound is a compound that has no effect on oxygen utilization efficiency. In some embodiments, the compound is compared to a compound that is known to decrease oxygen utilization efficiency. In some embodiments, the positive control compound is dexpramipexole.

In some embodiments, the method comprises identifying a compound that binds to the mitochondrial ATP synthase complex. In some embodiments, the method comprises contacting a test compound with the mitochondrial ATP synthase complex and identifying the test compound as the compound that binds the mitochondrial ATP synthase complex. Methods of determining whether a compound can bind to another protein or protein complex (e.g. ATP synthase complex) are known to one of skill in the art. These methods can be adopted to determine the binding of a test compound to another protein or protein complex, such as the ATP synthase complex.

In some embodiments, the mitochondria used in a method described herein are isolated mitochondria. An “isolated mitochondria” is a mitochondria that is has been isolated or purified from a cell. The mitochondria can be isolated to a 100% homogeneous composition where no other organelles are present. In some embodiments, the isolated mitochondria are not 100% homogeneous, but the mitochondria have been separated from the cell or are not encompassed by cell membrane or a lipid bilayer. In some embodiments, a cell comprises the mitochondria. In some embodiments, a cell comprises the mitochondrial ATP synthase complex. In some embodiments, an isolated mitochondria comprises the mitochondrial ATP synthase complex. The mitochondria used in methods described herein can be mitochondria isolated from a muscle cell or a central nervous system cell.

In some embodiments, the term “cell” refers to a muscle cell or a central nervous system cell. In some embodiments, a cell is a mammalian cell. In some embodiments, the cell is a human cell. In some embodiments, a cell is a cell that is or has been isolated from a diseased central nervous system (e.g. neurological) tissue or cell population. In some embodiments, the cell is an Alzheimer's central nervous system cell, a Parkinson's central nervous system cell, an Amyotrophic lateral sclerosis cell, a Huntingdon's disease cell, and the like. When a cell is referred to as being a certain disease or condition central nervous system cell it is to be understood that the cell is derived from or has been isolated from a patient or subject with the disease. The cell need not be a primary cell line. In some embodiments, the cell is a transformed cell. In some embodiments, the cell is not a transformed cell. In some embodiments, the cell is not actively dividing or is not capable of further cell division. In some embodiments, the cell is only capable of cell division if exposed to a mitogen (e.g. growth factor).

The ATP synthase complexes described herein can also be heterologously expressed. In some embodiments, the subunits necessary to form an ATP synthase complex are expressed in a cell or cell free system and the proteins are purified or isolated under conditions allowing the ATP synthase complex to form. The subunits can be expressed using routine recombinant techniques such as plasmids, vectors (e.g. viral or non-viral) and the like.

In some embodiments, a method is provided that identifies a compound that increases oxygen utilization efficiency and at least maintains ATP synthesis (e.g. production). In some embodiments, the method comprises identifying a compound that increases oxygen utilization efficiency as described herein. In some embodiments, the method comprises measuring ATP synthesis in the presence of the compound, wherein when the measured ATP synthesis is at least maintained in the presence of the compound indicates that the compound at least maintains ATP synthesis. A compound that increases oxygen utilization efficiency and at least maintains ATP synthesis is identified as a compound that increases oxygen utilization efficiency and at least maintains ATP synthesis. Measuring ATP synthesis can be by any method including, but not limited to, the methods described herein. For example, ATP synthesis can be measured using a luciferin-luciferase assay. The luciferin-luciferase assay measures ATP hydrolysis and can be correlated with the amount of ATP synthesis. A non-limiting example of this assay is described herein, but the conditions can be routinely modified to measure ATP synthesis and ATP. ATP synthesis can be also be measured using an NADH assay. In the NADH assay, for example, ATP hydrolysis can be measured using an NADH-ATP-synthase kit (Mitosciences, USA; catalog #MS541). The protocol can be modified according to the conditions. A non-limiting example of such a modification is described herein.

As described herein, in some embodiments, the compound (e.g. test compound) binds to an ATP synthase complex or is identified as binding to the ATP synthase complex. In some embodiments, the compounds binds to a F1 head, a b subunit, a OSCP subunit, or a combination thereof. In some embodiments, identifying a compound as a compound that binds the mitochondrial ATP synthase complex comprises determining whether the test compound can competitively inhibit the binding of a compound known to bind, or suspected of binding, to an ATP synthase complex. In some embodiments, the compound known to bind, or suspected of binding to an ATP synthase complex is dexpramipexole. The test compound or the compound known to, or suspected of binding, to an ATP synthase compound can have a detectable label. The detectable label can be used to determine whether the test compound can competitively inhibit the binding of another compound to an ATP synthase complex or whether the compound itself can bind to the ATP synthase complex even in the absence of the known compound. For example, if the test compound comprises a detectable label, the label's signal can change when the compound binds to an ATP synthase complex. In some embodiments, the label is not detectable until the compound binds to the ATP synthase complex. In some embodiments, the compound known to, or suspected of binding to an ATP synthase complex comprises a detectable label and the ability to detect the label is abrogated or diminished when the test compound is contacted with the ATP synthase complex. Other methods of detection can also be used. In some embodiments, the detectable label is a radioactive or fluorescent label.

Similarly to the measuring of the oxygen utilization efficiency, in some embodiments, the effect of the test compound on ATP synthesis can be compared to a positive or a negative control. With respect to ATP synthesis, a compound is said to be a positive control if it is known to at least maintain ATP synthesis. In some embodiments, the positive control increases ATP synthesis. With respect to ATP synthesis, a compound is said to be a negative control if it decreases ATP synthesis. In some embodiments, the positive control is dexpramipexole.

In some embodiments, the method comprises identifying a compound that enhances ATP synthesis greater than the positive control. In some embodiments, the identified compound enhances ATP synthesis more than dexpramipexole. A compound that increases ATP synthesis greater than a positive control is a compound that significantly increases ATP synthesis.

Methods of measuring oxygen utilization efficiency are described herein. In some embodiments, measuring oxygen utilization efficiency comprises measuring inhibition of mitochondrial conductance. In some embodiments, measuring oxygen utilization efficiency comprises measuring inhibition of mitochondrial conductance, which comprises measuring metabolic leak conductance. In some embodiments, measuring inhibition of mitochondrial conductance comprises a manual patch clamp recording and analysis. In some embodiments, measuring inhibition of mitochondrial conductance comprises automated patch clamp recording and analysis. In some embodiments, the automated patch clamp recording and analysis comprises a planar chip recording technique, which can also be referred to as planar patch clamp. The planar patch clamp technique is described, for example in Py et al (Biotechnology and Bioengineering, Volume 107, Issue 4, pages 593-600, 1 Nov. 2010), which is hereby incorporated by reference in its entirety. The planar patch clamp technique can be used, for example, to create a high throughput screen, so that many compounds can be tested and analyzed for their ability to increase oxygen utilization efficiency and/or increase ATP synthesis.

In some embodiments, the methods described herein comprise identifying a compound as a compound that treats a neurodegenerative disease. In some embodiments, the method comprises contacting a subject with a neurodegenerative disease with a compound identified that increases oxygen utilization efficiency, wherein a compound that improves the condition of the subject, inhibits the progression of the neurodegenerative disease, ameliorates the neurodegenerative disease indicates the compound as a compound that treats a neurodegenerative disease. In some embodiments, the method comprises testing a compound that has been identified as a compound that increases oxygen utilization efficiency and/or ATP synthesis using, for example, the methods described herein.

In some embodiments, a subject with a neurodegenerative disease that is described herein can be, for example, a mammal, a human, a non-human primate, a dog, a cat, a monkey, a mouse, a rat, a rodent, and the like. The neurodegenerative disease can be any including, but not limited to, those described herein.

In some embodiments, the methods described herein comprise identifying a compound as a compound that is a neuroprotectant. In some embodiments, the method comprises contacting a subject with a neurodegenerative disease with a compound that that increases oxygen utilization efficiency, wherein a compound that improves the condition of the subject inhibits the progression of the neurodegenerative disease, ameliorates the neurodegenerative disease indicates the compound as a compound that treats a neurodegenerative disease. In some embodiments, the method comprises testing a compound that has been identified as a compound that increases oxygen utilization efficiency and/or ATP synthesis using, for example, the methods described herein.

In some embodiments, the present invention provides methods of identifying a compound that increases oxygen utilization efficiency and at least maintains ATP synthesis. In some embodiments, the method comprises contacting a mitochondria or a sub-mitochondrial vesicle with a compound that binds to a mitochondrial ATP synthase complex and measuring oxygen utilization efficiency and ATP synthesis, wherein a compound that increases oxygen utilization efficiency and enhances or increases ATP synthesis identifies the compound as a compound that that increases oxygen utilization efficiency and at least maintains ATP synthesis. In some embodiments, the method comprises determining whether the compound can inhibit the binding of dexpramipexole to the mitochondrial ATP synthase complex, wherein a compound that inhibits the binding of a positive control (e.g. dexpramipexole) to the mitochondrial ATP synthase complex identifies the compound as a compound that binds to the mitochondrial synthase complex. In some embodiments, the method comprises comparing oxygen utilization efficiency and ATP synthesis in the presence of the compound to oxygen utilization efficiency and ATP synthesis in the presence of a positive control (e.g. dexpramipexole), wherein a compound that at least maintains oxygen utilization efficiency and at least maintains ATP synthesis as compared to a positive control (e.g. dexpramipexole) identifies the test compound as a compound that increases oxygen utilization efficiency and at least maintains ATP synthesis.

The positive or negative controls described herein can have a detectable label as described herein or with another detectable label. The specific detectable label is not significant and any label that can be detected either upon binding or inhibition of binding can be used.

In some embodiments, the methods provided by the present invention comprise identifying a compound as a compound that treats a neurodegenerative disease comprising contacting a subject with a neurodegenerative disease with a compound identified as a compound that that at least increases oxygen utilization efficiency and at least maintains ATP synthesis, wherein a compound that improves the condition of the subject; inhibits the progression of the neurodegenerative disease, or ameliorates the neurodegenerative disease indicates the compound as a compound that treats a neurodegenerative disease.

In some embodiments, the present invention provides a method of identifying a compound that treats a neurodegenerative disease. In some embodiments, the method comprises contacting a subject with a neurodegenerative disease with a test compound, wherein said test compound is a compound that increases oxygen utilization efficiency, a compound that at least maintains ATP synthesis, and/or a compound that binds to a mitochondrial ATP synthase complex, wherein a compound that improves the condition of the subject; inhibits the progression of the neurodegenerative disease, ameliorates the neurodegenerative disease indicates the compound as a compound that treats a neurodegenerative disease. In some embodiments, the method comprises identifying a compound that increases oxygen utilization efficiency, a compound that at least maintains ATP synthesis, and/or a compound that binds to a mitochondrial ATP synthase complex. In some embodiments, the test compound is a compound that increases oxygen utilization efficiency, at least maintains ATP synthesis, and binds to a mitochondrial ATP synthase complex.

The present invention also provides, in some embodiments, a method of treating a neurodegenerative disease in a subject comprising administering to the subject a compound identified by any of the methods described herein or a combination of any of the methods described herein. Additionally, any of the methods described herein can be combined in whole or in part with each method to produce a method of identifying a compound that increases oxygen utilization efficiency, at least maintains ATP synthesis, binds to an ATP synthesis complex, or any combination thereof.

Any test compound can be tested in the methods described herein. In some embodiments, the test compound or a compound used in a method described herein is not dexpramipexole.

In some embodiments, a method of identifying a neuroprotectant is provided. In some embodiments, the method comprises contacting a compound that binds a mitochondrial ATP synthase complex with a mitochondria or a sub-mitochondrial vesicle; and measuring oxygen utilization efficiency, wherein when the measured oxygen utilization efficiency in the mitochondria or a sub-mitochondrial vesicle increases in the presence of the compound that binds the mitochondrial ATP synthase complex indicates that the compound is a neuroprotectant. In some embodiments, the method comprises identifying a compound that binds to the mitochondrial ATP synthase complex. In some embodiments, the method comprises contacting a test compound with the mitochondrial ATP synthase complex; and identifying the test compound as the compound that binds the mitochondrial ATP synthase complex. In some embodiments, the method comprises measuring ATP synthesis in the presence of the compound, wherein when the measured ATP synthesis is at least maintained in the presence of the test compound which indicates that the compound is a neuroprotectant.

In some embodiments, the present invention provides a method of identifying a neuroprotectant. In some embodiments, the method comprises contacting a subject with a neurodegenerative disease with a test compound, wherein the test compound is a compound that increases oxygen utilization efficiency, a compound that at least maintains ATP synthesis, and/or a compound that binds to a mitochondrial ATP synthase complex, wherein a compound that improves the condition of the subject; inhibits the progression of the neurodegenerative disease, or ameliorates the neurodegenerative disease indicates the compound as a compound that treats a neurodegenerative disease.

In some embodiments, the present invention provides a method of identifying a compound that does not increase oxygen utilization efficiency comprising contacting a compound that binds a mitochondrial ATP synthase complex with a mitochondria or a sub-mitochondrial vesicle; and measuring oxygen utilization efficiency, wherein when the measured oxygen utilization efficiency in the mitochondria or a sub-mitochondrial vesicle is not increased in the presence of the compound that binds the mitochondrial ATP synthase complex indicates that the compound is a compound that does not increase oxygen utilization efficiency. In some embodiments, the method comprises measuring ATP synthesis in the presence of said the compound, wherein when the measured ATP synthesis is not maintained in the presence of the test compound indicates that the compound does not increase oxygen utilization efficiency and maintain ATP synthesis.

In some embodiments, a method is provided for identifying a compound that increases cell survival. In some embodiments, the method comprises contacting a cell with a compound that binds to an ATP synthase complex and increases oxygen utilization efficiency and measuring cell survival, wherein an increase in cell survival in the presence of the compound identifies the compound as a compound that increases cell survival. In some embodiments, the method comprises comparing the compound to a positive and/or a negative control. In some embodiments, the method comprises comparing the cell survival in the presence and the absence of the compound (e.g. test compound).

In some embodiments, the present invention provides methods for preparing a mitochondrial ATP synthase complex modulating compound comprising applying a three-dimensional molecular modeling algorithm to the atomic coordinates of at least a portion of the ATP synthase complex, particularly the F1 head; determining spatial coordinates of the at least a portion of the ATP synthase complex; electronically screening stored spatial coordinates of candidate compounds against the spatial coordinates of the at least a portion of the ATP synthase complex; identifying a compound that is substantially similar to the at least a portion of the ATP synthase complex; and synthesizing the identified compound. In certain embodiments, the ATP synthase complex includes its nine (9) subunits: the alpha (α), beta (β), b, c, delta (δ), d, epsilon (ε), gamma (γ), and OSCP (oligomycin-sensitivity conferring protein). In certain embodiments, the ATP synthase complex includes the b subunit and the OSCP subunit. In certain embodiments, the ATP synthase complex includes the b subunit. In certain embodiments, the ATP synthase complex includes the OSCP subunit.

In some embodiments, the present invention provides pharmaceutical compositions comprising an effective amount of a compound having a three-dimensional structure corresponding to atomic coordinates of at least a portion of a mitochondrial ATP synthase complex. In certain embodiments, the ATP synthase complex includes its nine (9) subunits: the alpha (α), beta (β), b, c, delta (δ), d, epsilon (ε), gamma (γ), and OSCP (oligomycin-sensitivity conferring protein). In certain embodiments, the ATP synthase complex includes the b subunit and the OSCP subunit. In certain embodiments, the ATP synthase complex includes the b subunit. In certain embodiments, the ATP synthase complex includes the OSCP subunit.

In some embodiments, the present invention provides methods and systems for identifying mitochondrial ATP synthase complex modulators comprising: a processor; and a processor readable storage medium in communication with the processor readable storage medium comprising the atomic coordinates of at least a portion of a mitochondrial ATP synthase complex. In certain embodiments, the ATP synthase complex includes its nine (9) subunits: the alpha (α), beta (β), b, c, delta (δ), d, epsilon (ε), gamma (γ), and (oligomycin-sensitivity conferring protein). In certain embodiments, the ATP synthase complex includes the b subunit and the OSCP subunit. In certain embodiments, the ATP synthase complex includes the b subunit. In certain embodiments, the ATP synthase complex includes the OSCP subunit.

In some embodiments, the present invention provides mitochondrial ATP synthase complex binding compounds comprising a molecule having a three-dimensional structure corresponding to atomic coordinates derived from at least a portion of an atomic model of the mitochondrial ATP synthase complex. In certain embodiments, the ATP synthase complex includes its nine (9) subunits: the alpha (α), beta (β), b, c, delta (δ), d, epsilon (ε), gamma (γ), and OSCP (oligomycin-sensitivity conferring protein). In certain embodiments, the ATP synthase complex includes the b subunit and the OSCP subunit. In certain embodiments, the ATP synthase complex includes the b subunit. In certain embodiments, the ATP synthase complex includes the OSCP subunit.

Recently, advances in protein crystallography and computational chemistry have introduced a new method of structure-based drug design into the field of drug development. X-ray crystallography (crystallography) is an established, well-studied technique that provides what can be best described as a three-dimensional picture of what a molecule looks like in a crystal. Scientists have used crystallography to solve the crystal structures for many biologically important molecules. Many classes of biomolecules can be studied by crystallography, including, but not limited to, proteins, DNA, RNA and viruses.

Crystallography has been used extensively to view ligand-protein complexes for structure-based drug design. To view such complexes, known ligands are usually soaked into the target molecule crystal, followed by crystallography of the complex. Sometimes, it is necessary to co-crystallize the ligands with the target molecule to obtain a suitable crystal. Given a “picture” of a target biomolecule or a ligand-protein complex, scientists can look for pockets or receptors where biological activity can take place. Thereafter, scientists can experimentally or computationally design high-affinity ligands (or drugs) for the protein/receptors. Computational methods have alternatively been used to screen for the binding of small molecules. This approach is also useful for developing new anti-mitotic agents.

Advantageous therapeutic embodiments would therefore comprise therapeutic and/or diagnostic agents based on or derived from the three-dimensional crystal structure of mitochondrial ATP synthase complex including novel binding sites that have one or more than one of the functional activities of mitochondrial ATP synthase complex. The ATP synthase complex may include its nine (9) subunits: the alpha (α), beta (β), b, c, delta (δ), d, epsilon (ε), gamma (γ), and OSCP (oligomycin-sensitivity conferring protein), or the ATP synthase complex may include the b subunit and the OSCP subunit, or the ATP synthase complex may include the b subunit or the ATP synthase complex may include the OSCP subunit.

Additional therapeutic embodiments would comprise therapeutic and/or diagnostic agents based on or derived from molecular modeling of other members of mitochondrial ATP synthase complex using the three-dimensional crystal structure of mitochondrial ATP synthase complex and its binding site provided herein.

As used herein, a “binding site” refers to a region of a molecule or molecular complex that, as a result of its shape and charge potential, favorably interacts or associates with another agent (including, without limitation, a protein, polypeptide, peptide, nucleic acid, including DNA or RNA, molecule, compound, antibody or drug) via various covalent and/or non-covalent binding forces. As used herein, the terms “bind” and “binding” when used to describe the interaction of a ligand with a binding site or a group of amino acids means that the binding site or group of amino acids are capable of forming a covalent or non-covalent bond or bonds with the ligand. Preferably, the binding between the ligand and the binding site or amino acid(s) is non-covalent. Such a non-covalent bond includes a hydrogen bond, an electrostatic bond, a van der Waals bond or the like. The binding of the ligand to the binding site may also be characterized by the ability of the ligand to co-crystallize with mitochondrial ATP synthase complex within the binding pocket of the instant invention. It is further understood that the use of the terms “bind” and “binding” when referring to the interaction of a ligand with the binding site of the instant invention includes the covalent or non-covalent interactions of the ligand with all or some of the amino acid residues comprising the binding site.

Consequently, an embodiment of the invention provides protein crystals of mitochondrial ATP synthase complex complexed with a ligand bound to the ligand binding site disclosed herein and methods for making mitochondrial ATP synthase complex or a mitochondrial ATP synthase complex homolog. Dexpramipexole is demonstrated to bind preferentially to the b and OSCP subunits of the ATP synthase complex, defining one or more binding sites that may serve as a substrate for the elucidation of the crystal structure of the protein-ligand interaction site(s). In certain embodiments, the ATP synthase complex includes its nine (9) subunits: the alpha (α), beta (β), b, c, delta (δ), d, epsilon (ε), gamma (γ), and OSCP (oligomycin-sensitivity conferring protein). In certain embodiments, the ATP synthase complex includes the b subunit and the OSCP subunit. In certain embodiments, the ATP synthase complex includes the b subunit. In certain embodiments, the ATP synthase complex includes the OSCP subunit.

The crystals provide means to obtain atomic modeling information of the specific amino acids and their atoms forming the binding site and that interact with molecules e.g., ligands or binding partners that bind to the mitochondrial ATP synthase complex, via the binding site.

The crystals also provide modeling information regarding the protein-ligand interaction, as well as the structure of ligands bound thereto. The mitochondrial ATP synthase complex crystal or a mitochondrial ATP synthase complex homolog according to the present invention can be obtained by crystallizing it with a material or compound or molecule which binds to the herein disclosed binding site of the mitochondrial ATP synthase complex. In certain embodiments, the ATP synthase complex includes its nine (9) subunits: the alpha (α), beta (β), b, c, delta (δ), d, epsilon (ε), gamma (γ), and OSCP (oligomycin-sensitivity conferring protein). In certain embodiments, the ATP synthase complex includes the b subunit and the OSCP subunit. In certain embodiments, the ATP synthase complex includes the b subunit. In certain embodiments, the ATP synthase complex includes the OSCP subunit.

In some embodiments, crystalline compositions of this invention are capable of diffracting X-rays to a resolution of better than about 3.5 A, and, in some embodiments, to a resolution of about 2.6 A or better, and even more preferably to a resolution of about 2.0 A or better, and are useful for determining the three-dimensional structure of the material. (The smaller the number of angstroms, the better the resolution).

In another aspect, the present invention provides the three-dimensional structure of human mitochondrial ATP synthase complex as well as the identification and characterization of a binding site there within. The identification of this site permits design and identification of compounds that bind to the ligand binding site and modulate mitochondrial ATP synthase complex related activities. The compounds include inhibitors which specifically inhibit cell proliferation.

Of equal import is the fact that knowledge of the three-dimensional structure of the binding site of mitochondrial ATP synthase complex provides a means for investigating the mechanism of action and tools for identifying modulators, including, but not limited to, inhibitors, of its function.

After the material designed by a computer is prepared and bound to mitochondrial ATP synthase complex to produce a crystal, the 3-dimensional structure of the complex may be determined at high enough resolution (over 0.28 nm) using X-ray crystallographic methods. The information gained therefrom e.g., about the interaction between—and the inhibitor obtained from this can then be used to modify the inhibitor and to increase the affinity of the inhibitor for the ligand binding site of mitochondrial ATP synthase complex. Thus, for example, those atoms considered to be involved in binding to the ligand binding site of mitochondrial ATP synthase complex disclosed herein can be mutated by exchanging one or more of the amino acid residues in the ligand binding site or in the motor domain that eventually effects the function of mitochondrial ATP synthase complex on the underlying cell.

Likewise, just as the three-dimensional modeling of mitochondrial ATP synthase complex is used, the coordinates from the X-ray defraction patterns can be either analyzed directly to provide the three-dimensional structure (if of sufficiently high resolution). Alternatively, the atomic coordinates for the crystallized mitochondrial ATP synthase complex, as provided herein, can be used for structure determination. The X-ray diffraction patterns obtained by methods of the present invention, can be provided on computer readable media, and used to provide electron density maps. The electron density maps, provided by analysis of the X-ray coordinates of mitochondrial ATP synthase complex complexed with Compound X, may then be fitted using suitable computer algorithms to generate secondary, tertiary and/or quaternary structures and/or domains of mitochondrial ATP synthase complex, which structures and/or domains are then used to provide an overall three-dimensional structure, as well as binding and/or active sites of mitochondrial ATP synthase complex.

As an example, the structure of renin has been modeled using the tertiary structure of endothiapepsin as a starting point for the derivation. Model building of cercarial elastase and tophozoite cysteine protease were each built from known serine and cysteine proteases that have less than 35% sequence identity. The resultant models were used to design inhibitors in the low micromolar range. (Proc. Natl. Acad. Sci. 1993, 90, 3583). Furthermore, alternative methods of tertiary structure determination that do not rely on X-ray diffraction techniques and thus do not require crystallization of the protein, such as NMR techniques, are simplified if a model of the structure is available for refinement using the additional data gathered by the alternative technique. Thus, knowledge of the tertiary structure of the mitochondrial ATP synthase complex binding site provides a significant window to the structure of the other family members. Thus, an embodiment of this invention envisions use of atomic coordinates of mitochondrial ATP synthase complex, or fragment, analog or variant thereof, to model a protein.

One skilled in the relevant art may use conventional molecular modeling methods to identify a ligand binding site of a mitochondrial ATP synthase complex of another species. Specifically, coordinates provided by the present invention may be used to characterize a three-dimensional structure of the target mitochondrial ATP synthase complex liganded or unliganded. Importantly, such a skilled artisan may, from such a structure, computationally visualize a putative binding site and identify and characterize other features based upon the coordinates provided herein. Such putative ligand binding sites may be further refined using chemical shift perturbations of spectra generated from various and distinct mitochondrial ATP synthase complex complexes, e.g. from other species, competitive and non-competitive inhibition experiments, and/or by the generation and characterization of mitochondrial ATP synthase complex or ligand mutants to identify critical residues or characteristics of the ligand binding site. Such identification of a putative ligand binding site is of great import in rational drug design.

One can use a method for determining the molecular structure of a molecular complex whose structure is unknown, comprising the steps of obtaining the molecular complex whose structure is unknown, e.g., from a related species, and then generating NMR data there from. The NMR data from the molecular complex whose structure is unknown can then be compared to the structure data obtained from the mitochondrial ATP synthase complex of the present invention. Then, 2D, 3D and 4D isotope filtering, editing and triple resonance NMR techniques can be used to conform the 3D structure described herein for the mitochondrial ATP synthase complex complexes to the NMR data from unknown target molecular complex. Alternatively, molecular replacement may be used to conform the 3D structure of the present invention to X-ray diffraction data from crystals of the unknown target molecular complex.

As a general rule, one may use knowledge of the geography of the various regions of the ligand binding site disclosed herein, e.g. hydrophobic and/or hydrophilic to design mitochondrial ATP synthase complex analogs (mutant) in which the overall mitochondrial ATP synthase complex structure is not changed, but change does affect biological activity (“biological activity” being used here in its broadest sense to denote function). Thus, one may make changes to the amino acid sequences to effectively obtain a mitochondrial ATP synthase complex analog/mutant that exhibits a greater affinity for its binding ligand. As well, one may correlate biological activity to structure. If the structure is not changed, and the mutation has no effect on biological activity, then the mutation has no biological function. If, however, the structure is not changed and the mutation does affect biological activity, then the residue (or atom) is essential to at least one biological function.

In other embodiments, such as through the use of computer simulation, the three dimensional (3D) surface structure of ATP synthase can be used as a target for predicting drug design. In this way modulating compounds can be found through computer assisted searches of databases. Compounds which contain the best predicted fit can then be visually inspected and tested under in vitro and in vivo conditions. This method also allows for the tinkering of the compound's structure to allow for optimal binding capacity, i.e., by testing the activity of analogs of the identified compounds in in vitro assays.

In one exemplary embodiment, the present invention provides a method for computational processing of a database containing three-dimensional structures of a large number of chemical compounds to identify compounds having high predicted binding affinity to a host molecule. The predicted binding affinity is validated through in vitro testing. One or more of the compounds having a binding affinity validated in vitro are further tested in vivo to provide a group of pharmacophores capable of having therapeutic activity involving the host molecule.

Computationally predicting a compound's binding affinity to a host molecule involves utilizing the three dimensional (3-D) structures of the host and the compound. As indicated above, the 3-D structure of the compound is obtained from a database of chemical compounds. The 3-D structure of the host protein can also be obtained from a protein database. However, in spite of important increases in the number of proteins having available 3-D structures, that number only covers a very small fraction of proteins having known biological function. Therefore, the invention includes a method for modeling the 3-D structure of the host protein, when such structure is not available.) Modeling the 3-D structure of the host protein includes obtaining the primary and secondary structures of the protein. Screening a database containing proteins having known 3-D structures, and retrieving from the database the structure of a protein having primary and/or secondary structures having a high degree of homology with the primary and/or secondary structures of the host molecule. The screening and selection methods are performed using one of the available homology screening computer programs. One example of a computer program capable of identifying a homologous protein of known 3-D structure is provided in the software package BLAST. BLAST can be accessed at http://www.ncbi.nlm.nih.gov/BLAST-/. The methodology utilized in BLAST is described in “Protein sequence similarity searches using patterns as seeds”, by Zheng Zhang, Alejandro A. Schffer, Webb Miller, Thomas L. Madden, David J. Lipman, Eugene V. Koonin, and Stephen F. Altchsul (1998), Nucleic Acids Res. 26:3986-3990, the contents of which are incorporated herein by reference in their entirety.

A template 3-D structure of the host protein is obtained through the program MODELLER. MODELLER can be obtained from Professor Andrej Sali, the Rockefeller University, 1230 York Avenue, New York, N.Y. 10021-6399. The methodology utilized in MODELLER is described in “Evaluation of comparative protein modeling by MODELLER” by Sali, A., Potterton, L., Yuan, F., van Vlijmen, H., & Karplus, M. (1995). Proteins, 23, 318-326, the contents of which are incorporated herein by reference in their entirety.

In forming a template 3-D structure of the target (i.e., mitochondrial ATP synthase complex or a portion thereof), each atom in of the backbone of the protein is assigned a position corresponding to the equivalent backbone atom of the homologous protein. Similarly, each atom of a side chain of the host protein having an equivalent side chain in the homologous protein is assigned the position corresponding to the position of the atom in the equivalent side chain of the homologous protein. The atom positions for the side chains not having an equivalent in the homologous protein are determined by constructing the side chain according to preferred internal coordinates and attaching the side chain to the backbone of the host protein. The template structure thus obtained is refined by minimizing the internal energy of the template protein. During the refinement, the positions of the atoms of the side chains having no equivalents in the homologous protein are adjusted while keeping the rest of the atoms of the template protein in a fixed position. This allows the atoms of the constructed side chains to adapt their positions to the part of the template structure determined by homology. The full template structure is then minimized (relaxed) by allowing all the atoms to move. Relaxing the template 3-D structure of the protein eliminates unfavorable contacts between the atoms of the protein and reduces the strain in the template 3-D structure.

The minimization of the energy function associated with the template structure can be performed by any minimization technique. In some embodiments, a minimization technique involves simulated annealing. This technique is incorporated in numerous commercial and non-commercial computer programs. One such computer program is included in the software package CHARMM. CHARMM can be obtained either from Dr. Martin Karplus at the Harvard University for academic users or from the Molecular Simulation Inc., San Diego, Calif. The simulated annealing methodology incorporated in CHARMM is described in “A program for macromolecular energy minimization, and dynamics calculations” by Brooks, B. R., Bruccoleri, R. E., Olafson, B. D., States, D. J., Swaminathan, S., and Karplus, M., J. Comp. Chem. 1 (1983) 187, the contents of which are incorporated herein by reference in their entirety.

Based on the refined structure of the host protein, a host-guest complex is formed by disposing a compound from the database in a receptor site of the protein.

The structure of the host-guest complex is defined by the position occupied by each atom in the complex in a three dimensional referential. The position of each atom is defined by a set of three coordinates in the referential. The structure of the host-guest complex is incorporated in a computer program capable of determining the degree of geometrical fit between the guest and the host in the complex. Programs based on shape complementarity can effectively rank guest-host complexes based on the geometrical fit between the host and the guest. In some embodiments, a program for ranking guest-host complexes based on the geometrical fit is provided in the software package DOCK. DOCK can be obtained from Dr. Irwin Kuntz at the Department of Pharmaceutical Chemistry, University of California at San Francisco, USA. The shape complementarity methodology of DOCK is described in “Critical evaluation of search algorithms used in automated molecular docking” by Ewing, T. J. A., and Kuntz, I. D. J. Comput. Chem. 18(9): 1175-1189, 1997, the contents of which are incorporated herein by reference in their entirety.

A group of compounds is extracted from the compound database for further processing based on their geometry fit rank. The compounds in the group have a guest-host complex geometrical fit of a predetermined rank or higher. The number of compounds in the geometry fit group is generally a small fraction of the total number of compounds in the database

For each compound in the geometry fit group, a predicted binding affinity to the receptor site of the host protein is determined by minimizing an energy function describing the interactions between the atoms of the compound and those of the protein. The minimization of the energy function is conducted by changing the position of the compound such that a guest-host complex structure corresponding to a minimum of the energy function is obtained.

The energy function includes energy terms describing non-bonded interactions between the atoms of the compound and those of the protein. The non-bonded energy terms include a term for atom-atom Van der Waals interactions and a term for charge-charge electrostatic interactions. The energy function does not include constraints on torsional degrees of freedom of the compound which provides greater flexibility in changing the position and conformation of the compound in the receptor site of the protein. A minimum energy value is obtained for each compound-protein complex.

Allowing for torsional flexibility in refining the structure of the complex greatly enhances the accuracy of the predicted binding energy of the complex. In this regard, a flexible compound can adopt a larger number of conformations inside the receptor site, thus allowing for probing a larger number of complex structures. Increasing the number of probable complex structures increases the probability of identifying a global minimum of the energy function. That is, a minimum having an energy lower than the energy associated with one or more other identified minima of the energy function (local minima). Identifying a global minimum for a given complex is greatly advantageous in that a more accurate predicted binding affinity is obtained for the complex. Increasing the accuracy of the predicted binding affinity increases the accuracy in energy based discrimination between the compounds of the geometry fit group, thus providing the best candidates for in vitro testing.

Several computational techniques have been previously used in adjusting the position of a guest in relation to a host. However, conventional programs based on those techniques do not provide satisfactory torsional flexibility in moving the guest within the receptor site of the host. Therefore, a new approach is provided for effectively including torsional energy in refining the position of the compound in the complex.

The complex energy minimization employs a non-conventional Monte Carlo simulation technique. The methodology incorporated in MCDOCK is described in “MCDOCK: A Monte Carlo simulation approach to the molecular docking problem” by Ming Liu and Shaomeng Wang, to be published in Journal of Computer-Aided Molecular Design, the contents of which are incorporated herein by reference in their entirety.

MCDOCK provides a minimization method based on a non-conventional Monte Carlo simulation technique which allows greater probability to reach a global energy minimum. In particular, the program only constrains the bonds and bond angles describing the structure of the guest host complex. Otherwise, the atoms are allowed to move freely in a force field determined by an energy function formed by Van der Waals and electrostatic terms only. This flexibility allows the guest to adopt various conformations within the receptor site of the host and thus explore a larger portion of the receptor site. This in turn allows the exploration of global minima, which improves the equality of the energy based binding affinity prediction.

The compounds in the geometry fit group are processed through MCDOCK such that for each compound, a compound-protein complex of minimum “MCDOCK” energy is determined. The compounds are then ranked according to the minimum energy obtained. A subgroup of compounds associated with complexes having a minimum energy lower than a predetermined energy value is formed. The number of compounds in the subgroup is also a small fraction of the total number of compounds in the geometry fit group.

The binding information associated with each compound in the subgroup is further refined by displaying on a computer screen an image of the complex structure of minimum energy. Displaying the compound-protein complex is conducted through one of the conventional chemical structure graphic visualization tools. In some embodiments, a graphic visualization tool is provided in the software package QUANTA (MOLECULAR SIMULATIONS, San Diego, Calif.).

The displayed complexes are visually examined to form a group of candidate compounds for in vitro testing. For example, the complexes are inspected for visual determination of the quality of docking of the compound into the receptor site of the protein. Visual inspection provides an effective basis for identifying compounds for in vitro testing. It should be noted that such visual inspection is impractical without the effective pruning of the compounds of the initial database provided by the pruning based on the combination of the geometry fit and complex energy minimization. Therefore, the number of compounds in the group discarded in the visual pruning step is much smaller than the number of compounds discarded in the geometry fit and energy based pruning

After putative binding compounds have been identified, the ability of such compounds to specifically bind to a particular receptor moiety, e.g., a specific ATP synthase or portion thereof, can be confirmed in vitro.

Methods for determining whether a compound binds to or interacts with to a particular target, i.e., target binding assays are well known in the art. In particular, this can be effected by use of competition assays. In general, this will involve providing a source of the particular receptor, a moiety known to interact with such receptor, e.g., peptide, and a compound, the receptor binding of which is to be tested. Compounds which bind the receptor will inhibit the binding of the other moiety, e.g., peptide, that is known to specifically bind said receptor.

Also, in the case of putative ATP synthase modulating compounds, these compounds can be tested in functional assays.

In further embodiments, compounds that modulate ATP synthase activity may be useful in treating diseases and conditions associated with or involving decreased mitochondrial function or mitochondrial dysfunction.

In further embodiments, compounds that increase mitochondrial bioenergetic efficiency may be useful in treating diseases and conditions associated with or involving decreased mitochondrial function or mitochondrial dysfunction.

In certain embodiments, such diseases and conditions include, but are not limited to, age-related macular degeneration, type II diabetes, skin diseases and disorders, coronary and cardiovascular diseases and disorders, inflammatory disorders and neurodegenerative diseases.

In various embodiments, compounds that modulate ATP synthase activity may be useful in treating a neurodegenerative disease. Non-limiting examples of neurodegenerative diseases include Huntington's Chorea, metabolically induced neurological damage, Alzheimer's disease, senile dementia, age associated cognitive dysfunction, vascular dementia, multi-infarct dementia, Lewy body dementia, neurodegenerative dementia, neurodegenerative movement disorder, ataxia, Friedreich's ataxia, multiple sclerosis, spinal muscular atrophy, primary lateral sclerosis, seizure disorders, motor neuron disorder or disease, inflammatory demyelinating disorder, Parkinson's disease, amyotrophic lateral sclerosis (ALS), hepatic encephalopathy, and chronic encephalitis. Thus, the compositions and methods of the invention may be used to treat nearly any individual exhibiting symptoms of a neurological disease or susceptible to such diseases.

The amount of the compound may vary. For example, in some embodiments, the amount of the compound may be from about 25 mg to about 1,000 mg, about 50 mg to about 1,000 mg, from about 100 mg to about 1,000 mg, from about 300 mg to about 1,000 mg, from about 500 mg to about 1,000 mg, and in certain embodiments, the amount may be from about 60 mg to about 300 mg.

In some embodiments, the amount of the compound administered per day, the therapeutically effective amount per day per kg, may be from about 2 mg/kg/day to about 1000 mg/kg/day, from about 4 mg/kg/day to about 1000 mg/kg/day, from about 2 mg/kg/day to about 500 mg/kg/day, from about 4 mg/kg/day to about 500 mg/kg/day, from about 2 mg/kg/day to about 200 mg/kg/day, or from about 4 mg/kg/day to about 200 mg/kg/day, and in other embodiments, the amount may be from about from about 1 mg/kg/day to about 100 mg/kg/day, from about 2 mg/kg/day to about 50 mg/kg/day, or from about 2 mg/kg/day to about 10 mg/kg/day. Thus, the daily dose administered to a patient, in various embodiments, may from about 100 mg/day to about 1000 mg/day, about 150 mg/day to 500 mg/day, and in particular embodiments, from about 150 mg/day to about 300 mg/day.

For example, in some aspects, the invention is directed to a pharmaceutical composition comprising a compound, as defined above, and a pharmaceutically acceptable carrier or diluent, or an effective amount of a pharmaceutical composition comprising a compound as defined above.

The compounds of the present invention can be administered in the conventional manner by any route where they are active. Administration can be systemic, topical, or oral. For example, administration can be, but is not limited to, parenteral, subcutaneous, intravenous, intramuscular, intraperitoneal, transdermal, oral, buccal, or ocular routes, or intravaginally, by inhalation, by depot injections, or by implants. Thus, modes of administration for the compounds of the present invention (either alone or in combination with other pharmaceuticals) can be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams.

Specific modes of administration will depend on the indication. The selection of the specific route of administration and the dose regimen is to be adjusted or titrated by the clinician according to methods known to the clinician in order to obtain the optimal clinical response. The amount of compound to be administered is that amount which is therapeutically effective. The dosage to be administered will depend on the characteristics of the subject being treated, e.g., the particular animal treated, age, weight, health, types of concurrent treatment, if any, and frequency of treatments, and can be easily determined by one of skill in the art (e.g., by the clinician).

Pharmaceutical formulations containing the compounds of the present invention and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder; comprising an effective amount of a polymer or copolymer of the present invention. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted.

The compounds of the present invention can be formulated for parenteral administration by injection, e.g., by bolus injection or continuous infusion. The compounds can be administered by continuous infusion subcutaneously over a period of about 15 minutes to about 24 hours. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

For oral administration, the compounds can be formulated readily by combining these compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compositions can take the form of, e.g., tablets or lozenges formulated in a conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The compounds of the present invention can also be formulated in rectal compositions such as suppositories or retention enemas, e.g., containing conventional suppository bases such as cocoa butter or other glycerides.

In addition to the formulations described previously, the compounds of the present invention can also be formulated as a depot preparation. Such long acting formulations can be administered by implantation (for example subcutaneously or intramuscularly) or by intramuscular injection.

Depot injections can be administered at about 1 to about 6 months or longer intervals. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

In transdermal administration, the compounds of the present invention, for example, can be applied to a plaster, or can be applied by transdermal, therapeutic systems that are consequently supplied to the organism.

Pharmaceutical compositions of the compounds also can comprise suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols.

The compounds of the present invention can also be administered in combination with other active ingredients, such as, for example, adjuvants, protease inhibitors, or other compatible drugs or compounds where such combination is seen to be desirable or advantageous in achieving the desired effects of the methods described herein.

In some embodiments, the disintegrant component comprises one or more of croscarmellose sodium, carmellose calcium, crospovidone, alginic acid, sodium alginate, potassium alginate, calcium alginate, an ion exchange resin, an effervescent system based on food acids and an alkaline carbonate component, clay, talc, starch, pregelatinized starch, sodium starch glycolate, cellulose floc, carboxymethylcellulose, hydroxypropylcellulose, calcium silicate, a metal carbonate, sodium bicarbonate, calcium citrate, or calcium phosphate.

In some embodiments, the diluent component comprises one or more of mannitol, lactose, sucrose, maltodextrin, sorbitol, xylitol, powdered cellulose, microcrystalline cellulose, carboxymethylcellulose, carboxyethylcellulose, methylcellulose, ethylcellulose, hydroxyethylcellulose, methylhydroxyethylcellulose, starch, sodium starch glycolate, pregelatinized starch, a calcium phosphate, a metal carbonate, a metal oxide, or a metal aluminosilicate.

In some embodiments, the optional lubricant component, when present, comprises one or more of stearic acid, metallic stearate, sodium stearyl fumarate, fatty acid, fatty alcohol, fatty acid ester, glyceryl behenate, mineral oil, vegetable oil, paraffin, leucine, silica, silicic acid, talc, propylene glycol fatty acid ester, polyethoxylated castor oil, polyethylene glycol, polypropylene glycol, polyalkylene glycol, polyoxyethylene-glycerol fatty ester, polyoxyethylene fatty alcohol ether, polyethoxylated sterol, polyethoxylated castor oil, polyethoxylated vegetable oil, or sodium chloride.

As used herein, the term “alginic acid” refers to a naturally occurring hydrophilic colloidal polysaccharide obtained from the various species of seaweed, or synthetically modified polysaccharides thereof.

As used herein, the term “sodium alginate” refers to a sodium salt of alginic acid and can be formed by reaction of alginic acid with a sodium containing base such as sodium hydroxide or sodium carbonate. As used herein, the term “potassium alginate” refers to a potassium salt of alginic acid and can be formed by reaction of alginic acid with a potassium containing base such as potassium hydroxide or potassium carbonate. As used herein, the term “calcium alginate” refers to a calcium salt of alginic acid and can be formed by reaction of alginic acid with a calcium containing base such as calcium hydroxide or calcium carbonate. Suitable sodium alginates, calcium alginates, and potassium alginates include, but are not limited to, those described in R. C. Rowe and P. J. Shesky, Handbook of pharmaceutical excipients, (2006), 5th ed., which is incorporated herein by reference in its entirety. Suitable sodium alginates, include, but are not limited to, Kelcosol (available from ISP), Kelfone LVCR and HVCR (available from ISP), Manucol (available from ISP), and Protanol (available from FMC Biopolymer).

As used herein, the term “calcium silicate” refers to a silicate salt of calcium.

As used herein, the term “calcium phosphate” refers to monobasic calcium phosophate, dibasic calcium phosphate or tribasic calcium phosphate.

Cellulose, cellulose floc, powdered cellulose, microcrystalline cellulose, silicified microcrystalline cellulose, carboxyethyl cellulose, carboxymethylcellulose, hydroxyethylcellulose, methylhydroxyethylcellulose, hydroxymethyl cellulose, hydroxypropylcellulose, hydroxypropylmethylcellulose, hydroxypropylmethylcellulose phthalate, ethylcellulose, methylcellulose, carboxymethylcellulose sodium, and carboxymethyl cellulose calcium include, but are not limited to, those described in R. C. Rowe and P. J. Shesky, Handbook of pharmaceutical excipients, (2006), 5th ed., which is incorporated herein by reference in its entirety. As used herein, cellulose refers to natural cellulose. The term “cellulose” also refers to celluloses that have been modified with regard to molecular weight and/or branching, particularly to lower molecular weight. The term “cellulose” further refers to celluloses that have been chemically modified to attach chemical functionality such as carboxy, hydroxyl, hydroxyalkylene, or carboxyalkylene groups. As used herein, the term “carboxyalkylene” refers to a group of formula -alkylene-C(O)OH, or salt thereof. As used herein, the term “hydroxyalkylene” refers to a group of formula -alkylene-OH.

Suitable powdered celluloses for use in the invention include, but are not limited to Arbocel (available from JRS Pharma), Sanacel (available from CFF GmbH), and Solka-Floc (available from International Fiber Corp.).

Suitable microcrystalline celluloses include, but are not limited to, the Avicel pH series (available from FMC Biopolymer), Celex (available from ISP), Celphere (available from Asahi Kasei), Ceolus KG (available from Asahi Kasei), and Vivapur (available from JRS Pharma).

As used herein, the term “silicified microcrystalline cellulose” refers to a synergistic intimate physical mixture of silicon dioxide and microcrystalline cellulose. Suitable silicified microcrystalline celluloses include, but are not limited to, ProSolv (available from JRS Pharma).

As used herein, the term “carboxymethylcellulose sodium” refers to a cellulose ether with pendant groups of formula Na^{+ -}O—C(O)—CH₂—, attached to the cellulose via an ether linkage. Suitable carboxymethylcellulose sodium polymers include, but are not limited to, Akucell (available from Akzo Nobel), Aquasorb (available from Hercules), Blanose (available from Hercules), Finnfix (available from Noviant), Nymel (available from Noviant), and Tylose CB (available from Clariant).

As used herein, the term “carboxymethylcellulose calcium” refers to a cellulose ether with a pendant groups of formula —CH₂—O—C(O)—O⁻½ Ca²⁺, attached to the cellulose via an ether linkage.

As used herein, the term “carboxymethylcellulose” refers to a cellulose ether with pendant carboxymethyl groups of formula HO—C(O)—CH₂—, attached to the cellulose via an ether linkage. Suitable carboxymethylcellulose calcium polymers include, but are not limited to, Nymel ZSC (available from Noviant).

As used herein, the term “carboxyethylcellulose” refers to a cellulose ether with pendant carboxymethyl groups of formula HO—C(O)—CH₂—CH₂—, attached to the cellulose via an ether linkage.

As used herein, the term “hydroxyethylcellulose” refers to a cellulose ether with pendant hydroxyethyl groups of formula HO—CH₂—CH₂—, attached to the cellulose via an ether linkage. Suitable hydroxyethylcelluloses include, but are not limited to, Cellosize HEC (available from DOW), Natrosol (available from Hercules), and Tylose PHA (available from Clariant).

As used herein, the term “methylhydroxyethylcellulose” refers to a cellulose ether with pendant methyloxyethyl groups of formula CH₃—O—CH₂—CH₂—, attached to the cellulose via an ether linkage. Suitable methylhydroxyethylcelluloses include, but are not limited to, the Culminal MHEC series (available from Hercules), and the Tylose series (available from Shin Etsu).

As used herein, the term “hydroxypropylcellulose”, or “hypomellose”, refers a cellulose that has pendant hydroxypropoxy groups, and includes both high- and low-substituted hydroxypropylcellulose. In some embodiments, the hydroxypropylcellulose has about 5% to about 25% hydroxypropyl groups. Suitable hydroxypropylcelluloses include, but are not limited to, the Klucel series (available from Hercules), the Methocel series (available from Dow), the Nisso HPC series (available from Nisso), the Metolose series (available from Shin Etsu), and the LH series, including LHR-11, LH-21, LH-31, LH-20, LH-30, LH-22, and LH-32 (available from Shin Etsu).

As used herein, the term “methyl cellulose” refers to a cellulose that has pendant methoxy groups. Suitable methyl celluloses include, but are not limited to Culminal MC (available from Hercules).

As used herein, the term “ethyl cellulose” refers to a cellulose that has pendant ethoxy groups. Suitable ethyl celluloses include, but are not limited to Aqualon (available from Hercules).

As used herein, the term “carmellose calcium” refers to a crosslinked polymer of carboxymethylcellulose calcium.

As used herein, the term “croscarmellose sodium” refers to a crosslinked polymer of carboxymethylcellulose sodium.

As used herein, the term “crospovidone” refers to a crosslinked polymer of polyvinylpyrrolidone. Suitable crospovidone polymers include, but are not limited to Polyplasdone XL-10 (available from ISP) and Kollidon CL and CL-M (available from BASF).

As used herein, the term “crosslinked poly(acrylic acid)” refers to a polymer of acrylic acid which has been crosslinked. The crosslinked polymer may contain other monomers in addition to acrylic acid. Additionally, the pendant carboxy groups on the crosslinked polymer may be partially or completely neutralized to form a pharmaceutically acceptable salt of the polymer. In some embodiments, the crosslinked poly(acrylic acid) is neutralized by ammonia or sodium hydroxide. Suitable crosslinked poly(acrylic acid) polymers include, but are not limited to, the Carbopol series (available from Noveon).

As used herein, the term “an effervescent system based on food acids and an alkaline carbonate component” refers to a excipient combination of food acids and alkaline carbonates that releases carbon dioxide gas when administered. Suitable effervescent systems are those that those utilizing food acids (such as citric acid, tartaric acid, malic acid, fumaric acid, lactic acid, adipic acid, ascorbic acid, aspartic acid, erythorbic acid, glutamic acid, and succinic acid) and an alkaline carbonate component (such as sodium bicarbonate, calcium carbonate, magnesium carbonate, potassium carbonate, ammonium carbonate, etc.).

As used herein, the term “fatty acid”, employed alone or in combination with other terms, refers to an aliphatic acid that is saturated or unsaturated. In some embodiments, the fatty acid in a mixture of different fatty acids. In some embodiments, the fatty acid has between about eight to about thirty carbons on average. In some embodiments, the fatty acid has about eight to about twenty-four carbons on average. In some embodiments, the fatty acid has about twelve to about eighteen carbons on average. Suitable fatty acids include, but are not limited to, stearic acid, lauric acid, myristic acid, erucic acid, palmitic acid, palmitoleic acid, capric acid, caprylic acid, oleic acid, linoleic acid, linolenic acid, hydroxystearic acid, 12-hydroxystearic acid, cetostearic acid, isostearic acid, sesquioleic acid, sesqui-9-octadecanoic acid, sesquiisooctadecanoic acid, benhenic acid, isobehenic acid, and arachidonic acid, or mixtures thereof.

As used herein, the term “fatty acid ester” refers to a compound formed between a fatty acid and a hydroxyl containing compound. In some embodiments, the fatty acid ester is a sugar ester of fatty acid. In some embodiments, the fatty acid ester is a glyceride of fatty acid. In some embodiments, the fatty acid ester is an ethoxylated fatty acid ester.

As used herein, the term “fatty alcohol”, employed alone or in combination with other terms, refers to an aliphatic alcohol that is saturated or unsaturated. In some embodiments, the fatty alcohol in a mixture of different fatty alcohols. In some embodiments, the fatty alcohol has between about eight to about thirty carbons on average. In some embodiments, the fatty alcohol has about eight to about twenty-four carbons on average. In some embodiments, the fatty alcohol has about twelve to about eighteen carbons on average. Suitable fatty alcohols include, but are not limited to, stearyl alcohol, lauryl alcohol, palmityl alcohol, palmitolyl acid, cetyl alcohol, capryl alcohol, caprylyl alcohol, oleyl alcohol, linolenyl alcohol, arachidonic alcohol, behenyl alcohol, isobehenyl alcohol, selachyl alcohol, chimyl alcohol, and linoleyl alcohol, or mixtures thereof.

As used herein, the term “ion-exchange resin” refers to an ion-exchange resin that is pharmaceutically acceptable and that can be weakly acidic, weakly basic, strongly acidic or strongly basic. Suitable ion-exchange resins include, but are not limited to Amberlite™ IRP64, IRP88 and IRP69 (available from Rohm and Haas) and Duolite™ AP143 (available from Rohm and Haas). In some embodiments, the ion-exchange resin is a crosslinked polymer resin comprising acrylic acid, methacrylic acid, or polystyrene sulfonate, or salts thereof. In some embodiments, the ion-exchange resin is polacrilex resin, polacrilin potassium resin, or cholestyramine resin.

Suitable mannitols include, but are not limited to, PharmMannidex (available from Cargill), Pearlitol (available from Roquette), and Mannogem (available from SPI Polyols).

As used herein, the term “metal aluminosilicate” refers to any metal salt of an aluminosilicate, including, but not limited to, magnesium aluminometasilicate. Suitable magnesium aluminosilicates include, but are not limited to Neusilin (available from Fuji Chemical), Pharmsorb (available from Engelhard), and Veegum (available from R.T. Vanderbilt Co., Inc.). In some embodiments, the metal aluminosilicate is bentonite.

As used herein, the term “metal carbonate” refers to any metallic carbonate, including, but not limited to sodium carbonate, calcium carbonate, and magnesium carbonate, and zinc carbonate.

As used herein, the term “metal oxide” refers to any metallic oxide, including, but not limited to, calcium oxide or magnesium oxide.

As used herein, the term “metallic stearate” refers to a metal salt of stearic acid. In some embodiments, the metallic stearate is calcium stearate, zinc stearate, or magnesium stearate. In some embodiments, the metallic stearate is magnesium stearate.

As used herein, the term “mineral oil” refers to both unrefined and refined (light) mineral oil. Suitable mineral oils include, but are not limited to, the Avatech™ grades (available from Avatar Corp.), Drakeol™ grades (available from Penreco), Sirius™ grades (available from Shell), and the Citation™ grades (available from Avater Corp.).

As used herein, the term “polyethoxylated castor oil”, refers to a compound formed from the ethoxylation of castor oil, wherein at least one chain of polyethylene glycol is covalently bound to the castor oil. The castor oil may be hydrogenated or unhydrogenated. Synonyms for polyethoxylated castor oil include, but are not limited to polyoxyl castor oil, hydrogenated polyoxyl castor oil, mcrogolglyceroli ricinoleas, macrogolglyceroli hydroxystearas, polyoxyl 35 castor oil, and polyoxyl 40 hydrogenated castor oil. Suitable polyethoxylated castor oils include, but are not limited to, the Nikkol™ HCO series (available from Nikko Chemicals Co. Ltd.), such as Nikkol HCO-30, HC-40, HC-50, and HC-60 (polyethylene glycol-30 hydrogenated castor oil, polyethylene glycol-40 hydrogenated castor oil, polyethylene glycol-50 hydrogenated castor oil, and polyethylene glycol-60 hydrogenated castor oil, Emulphor™ EL-719 (castor oil 40 mole-ethoxylate, available from Stepan Products), the Cremophore™ series (available from BASF), which includes Cremophore RH40, RH60, and EL35 (polyethylene glycol-40 hydrogenated castor oil, polyethylene glycol-60 hydrogenated castor oil, and polyethylene glycol-35 hydrogenated castor oil, respectively), and the Emulgin® RO and HRE series (available from Cognis PharmaLine). Other suitable polyoxyethylene castor oil derivatives include those listed in R. C. Rowe and P. J. Shesky, Handbook of pharmaceutical excipients, (2006), 5th ed., which is incorporated herein by reference in its entirety.

As used herein, the term “polyethoxylated sterol” refers to a compound, or mixture of compounds, derived from the ethoxylation of sterol molecule. Suitable polyethoyxlated sterols include, but are not limited to, PEG-24 cholesterol ether, Solulan™ C-24 (available from Amerchol); PEG-30 cholestanol, Nikkol™ DHC (available from Nikko); Phytosterol, GENEROL™ series (available from Henkel); PEG-25 phyto sterol, Nikkol™ BPSH-25 (available from Nikko); PEG-5 soya sterol, Nikkol™ BPS-5 (available from Nikko); PEG-10 soya sterol, Nikkol™ BPS-10 (available from Nikko); PEG-20 soya sterol, Nikkol™ BPS-20 (available from Nikko); and PEG-30 soya sterol, Nikkol™ BPS-30 (available from Nikko). As used herein, the term “PEG” refers to polyethylene glycol.

As used herein, the term “polyethoxylated vegetable oil” refers to a compound, or mixture of compounds, formed from ethoxylation of vegetable oil, wherein at least one chain of polyethylene glycol is covalently bound to the vegetable oil. In some embodiments, the fatty acids has between about twelve carbons to about eighteen carbons. In some embodiments, the amount of ethoxylation can vary from about 2 to about 200, about 5 to 100, about 10 to about 80, about 20 to about 60, or about 12 to about 18 of ethylene glycol repeat units. The vegetable oil may be hydrogenated or unhydrogenated. Suitable polyethoxylated vegetable oils, include but are not limited to, Cremaphor™ EL or RH series (available from BASF), Emulphor™ EL-719 (available from Stepan products), and Emulphor™ EL-620P (available from GAF).

As used herein, the term “polyethylene glycol” refers to a polymer containing ethylene glycol monomer units of formula —O—CH₂—CH₂—. Suitable polyethylene glycols may have a free hydroxyl group at each end of the polymer molecule, or may have one or more hydroxyl groups etherified with a lower alkyl, e.g., a methyl group. Also suitable are derivatives of polyethylene glycols having esterifiable carboxy groups. Polyethylene glycols useful in the present invention can be polymers of any chain length or molecular weight, and can include branching. In some embodiments, the average molecular weight of the polyethylene glycol is from about 200 to about 9000. In some embodiments, the average molecular weight of the polyethylene glycol is from about 200 to about 5000. In some embodiments, the average molecular weight of the polyethylene glycol is from about 200 to about 900. In some embodiments, the average molecular weight of the polyethylene glycol is about 400. Suitable polyethylene glycols include, but are not limited to polyethylene glycol-200, polyethylene glycol-300, polyethylene glycol-400, polyethylene glycol-600, and polyethylene glycol-900. The number following the dash in the name refers to the average molecular weight of the polymer. In some embodiments, the polyethylene glycol is polyethylene glycol-400. Suitable polyethylene glycols include, but are not limited to the Carbowax™ and Carbowax™ Sentry series (available from Dow), the Lipoxol™ series (available from Brenntag), the Lutrol™ series (available from BASF), and the Pluriol™ series (available from BASF).

As used herein, the term “polyoxyethylene-alkyl ether” refers to a monoalkyl or dialkylether of polyoxyethylene, or mixtures thereof. In some embodiments, the polyoxyethylene-alkyl ether is a polyoxyethylene fatty alcohol ether.

As used herein, the term “polyoxyethylene fatty alcohol ether” refers to an monoether or diether, or mixtures thereof, formed between polyethylene glycol and a fatty alcohol. Fatty alcohols that are useful for deriving polyoxyethylene fatty alcohol ethers include, but are not limited to, those defined herein. In some embodiments, the polyoxyethylene portion of the molecule has about 2 to about 200 oxyethylene units. In some embodiments, the polyoxyethylene portion of the molecule has about 2 to about 100 oxyethylene units. In some embodiments, the polyoxyethylene portion of the molecule has about 4 to about 50 oxyethylene units. In some embodiments, the polyoxyethylene portion of the molecule has about 4 to about 30 oxyethylene units. In some embodiments, the polyoxyethylene fatty alcohol ether comprises ethoxylated stearyl alcohols, cetyl alcohols, and cetylstearyl alcohols (cetearyl alcohols). Suitable polyoxyethylene fatty alcohol ethers include, but are not limited to, the Brij™ series of surfactants (available from Uniqema), which includes Brij 30, 35, 52, 56, 58, 72, 76, 78, 93Veg, 97, 98, and 721, the Cremophor™ A series (available from BASF), which includes Cremophor A6, A20, and A25, the Emulgen™ series (available from Kao Corp.), which includes Emulgen 104P, 123P, 210P, 220, 320P, and 409P, the Ethosperse™ (available from Lonza), which includes Ethosperse 1A4, 1A12, TDAa6, S120, and G26, the Ethylan™ series (available from Brenntag), which includes Ethylan D252, 253, 254, 256, 257, 2512, and 2560, the Plurafac™series (available from BASF), which includes Plurafac RA20, RA30, RA40, RA43, and RA340, the Ritoleth™ and Ritox™ series (available from Rita Corp.), the Volpo™ series (available from Croda), which includes Volpo N 10, N 20, S2, S10, C2, C20, CS10, CS20, L4, and L23, and the Texafor™ series, which includes Texafor A1P, AP, A6, A10, A14, A30, A45, and A60. Other suitable polyoxyethylene fatty alcohol ethers include, but are not limited to, polyethylene glycol (13)stearyl ether (steareth-13), polyethylene glycol (14)stearyl ether (steareth-14), polyethylene glycol (15)stearyl ether (steareth-15), polyethylene glycol (16)stearyl ether (steareth-16), polyethylene glycol (17)stearyl ether (steareth-17), polyethylene glycol (18)stearyl ether (steareth-18), polyethylene glycol (19)stearyl ether (steareth-19), polyethylene glycol (20)stearyl ether (steareth-20), polyethylene glycol (12)isostearyl ether (isosteareth-12), polyethylene glycol (13)isostearyl ether (isosteareth-13), polyethylene glycol (14)isostearyl ether (isosteareth-14), polyethylene glycol (15)isostearyl ether (isosteareth-15), polyethylene glycol (16)isostearyl ether (isosteareth-16), polyethylene glycol (17)isostearyl ether (isosteareth-17), polyethylene glycol (18)isostearyl ether (isosteareth-18), polyethylene glycol (19)isostearyl ether (isosteareth-19), polyethylene glycol (20)isostearyl ether (isosteareth-20), polyethylene glycol (13)cetyl ether (ceteth-13), polyethylene glycol (14)cetyl ether (ceteth-14), polyethylene glycol (15)cetyl ether (ceteth-15), polyethylene glycol (16)cetyl ether (ceteth-16), polyethylene glycol (17)cetyl ether (ceteth-17), polyethylene glycol (18)cetyl ether (ceteth-18), polyethylene glycol (19)cetyl ether (ceteth-19), polyethylene glycol (20)cetyl ether (ceteth-20), polyethylene glycol (13)isocetyl ether (isoceteth-13), polyethylene glycol (14)isocetyl ether (isoceteth-14), polyethylene glycol (15)isocetyl ether (isoceteth-15), polyethylene glycol (16)isocetyl ether (isoceteth-16), polyethylene glycol (17)isocetyl ether (isoceteth-17), polyethylene glycol (18)isocetyl ether (isoceteth-18), polyethylene glycol (19)isocetyl ether (isoceteth-19), polyethylene glycol (20)isocetyl ether (isoceteth-20), polyethylene glycol (12)oleyl ether (oleth-12), polyethylene glycol (13)oleyl ether (oleth-13), polyethylene glycol (14)oleyl ether (oleth-14), polyethylene glycol (15)oleyl ether (oleth-15), polyethylene glycol (12) lauryl ether (laureth-12), polyethylene glycol (12)isolauryl ether (isolaureth-12), polyethylene glycol (13)cetylstearyl ether (ceteareth-13), polyethylene glycol (14)cetylstearyl ether (ceteareth-14), polyethylene glycol (15)cetylstearyl ether (ceteareth-15), polyethylene glycol (16)cetylstearyl ether (ceteareth-16), polyethylene glycol (17)cetylstearyl ether (ceteareth-17), polyethylene glycol (18)cetylstearyl ether (ceteareth-18), polyethylene glycol (19)cetylstearyl ether (ceteareth-19), and polyethylene glycol (20)cetylstearyl ether (ceteareth-20). The numbers following the “polyethylene glycol” term refer to the number of oxyethylene repeat units in the compound. Blends of polyoxyethylene fatty alcohol ethers with other materials are also useful in the invention. A non-limiting example of a suitable blend is Arlacel™ 165 or 165 VEG (available from Uniqema), a blend of glycerol monostearate with polyethylene glycol-100 stearate. Other suitable polyoxyethylene fatty alcohol ethers include those listed in R. C. Rowe and P. J. Shesky, Handbook of pharmaceutical excipients, (2006), 5th ed., which is incorporated herein by reference in its entirety.

As used herein, the term “polyoxyethylene-glycerol fatty ester” refers to ethoxylated fatty acid ester of glycerine, or mixture thereof. In some embodiments, the polyoxyethylene portion of the molecule has about 2 to about 200 oxyethylene units. In some embodiments, the polyoxyethylene portion of the molecule has about 2 to about 100 oxyethylene units. In some embodiments, the polyoxyethylene portion of the molecule has about 4 to about 50 oxyethylene units. In some embodiments, the polyoxyethylene portion of the molecule has about 4 to about 30 oxyethylene units. Suitable polyoxyethylene-glycerol fatty esters include, but are not limited to, PEG-20 glyceryl laurate, Tagat™ L (Goldschmidt); PEG-30 glyceryl laurate, Tagat™ L2 (Goldschmidt); PEG-15 glyceryl laurate, Glycerox™ L series (Croda); PEG-40 glyceryl laurate, Glycerox™ L series (Croda); PEG-20 glyceryl stearate, Capmul™ EMG (ABITEC), Aldo MS-20 KFG (Lonza); PEG-20 glyceryl oleate, Tagat™ 0 (Goldschmidt); PEG-30 glyceryl oleate, Tagat™ O₂ (Goldschmidt).

As used herein, the term “propylene glycol fatty acid ester” refers to an monoether or diester, or mixtures thereof, formed between propylene glycol or polypropylene glycol and a fatty acid. Fatty acids that are useful for deriving propylene glycol fatty alcohol ethers include, but are not limited to, those defined herein. In some embodiments, the monoester or diester is derived from propylene glycol. In some embodiments, the monoester or diester has about 1 to about 200 oxypropylene units. In some embodiments, the polypropylene glycol portion of the molecule has about 2 to about 100 oxypropylene units. In some embodiments, the monoester or diester has about 4 to about 50 oxypropylene units. In some embodiments, the monoester or diester has about 4 to about 30 oxypropylene units. Suitable propylene glycol fatty acid esters include, but are not limited to, propylene glycol laurates: Lauroglycol™ FCC and 90 (available from Gattefosse); propylene glycol caprylates: Capryol™ PGMC and 90 (available from Gatefosse); and propylene glycol dicaprylocaprates: Labrafac™ PG (available from Gatefosse).

Suitable sorbitols include, but are not limited to, PharmSorbidex E420 (available from Cargill), Liponic 70-NC and 76-NC (available from Lipo Chemical), Neosorb (available from Roquette), Partech SI (available from Merck), and Sorbogem (available from SPI Polyols).

Starch, sodium starch glycolate, and pregelatinized starch include, but are not limited to, those described in R. C. Rowe and P. J. Shesky, Handbook of pharmaceutical excipients, (2006), 5th ed., which is incorporated herein by reference in its entirety.

As used herein, the term “starch” refers to any type of natural or modified starch including, but not limited to, maize starch (also known as corn starch or maydis amylum), potato starch (also known as solani amylum), rice starch (also known as oryzae amylum), wheat starch (also known as tritici amylum), and tapioca starch. The term “starch” also refers to starches that have been modified with regard to molecular weight and branching. The term “starch” further refers to starches that have been chemically modified to attach chemical functionality such as carboxy, hydroxyl, hydroxyalkylene, or carboxyalkylene groups. As used herein, the term “carboxyalkylene” refers to a group of formula -alkylene-C(O)OH, or salt thereof. As used herein, the term “hydroxyalkylene” refers to a group of formula -alkylene-OH. Suitable sodium starch glycolates include, but are not limited to, Explotab (available from JRS Pharma), Glycolys (available from Roquette), Primojel (available from DMV International), and Vivastar (available from JRS Pharma).

Suitable pregelatinized starches include, but are not limited to, Lycatab C and PGS (available from Roquette), Merigel (available from Brenntag), National 78-1551 (available from National Starch), Spress B820 (available from GPC), and Starch 1500 (available from Colorcon).

As used herein, the term “stearoyl macrogol glyceride” refers to a polyglycolized glyceride synthesized predominately from stearic acid or from compounds derived predominately from stearic acid, although other fatty acids or compounds derived from other fatty acids may used in the synthesis as well. Suitable stearoyl macrogol glycerides include, but are not limited to, Gelucire® 50/13 (available from Gattefosse).

As used herein, the term “vegetable oil” refers to naturally occurring or synthetic oils, which may be refined, fractionated or hydrogenated, including triglycerides. Suitable vegetable oils include, but are not limited to castor oil, hydrogenated castor oil, sesame oil, corn oil, peanut oil, olive oil, sunflower oil, safflower oil, soybean oil, benzyl benzoate, sesame oil, cottonseed oil, and palm oil. Other suitable vegetable oils include commercially available synthetic oils such as, but not limited to, Miglyol™ 810 and 812 (available from Dynamit Nobel Chicals, Sweden) Neobee™ M5 (available from Drew Chemical Corp.), Alofine™ (available from Jarchem Industries), the Lubritab™ series (available from JRS Pharma), the Sterotex™ (available from Abitec Corp.), Softisan™ 154 (available from Sasol), Croduret™ (available from Croda), Fancol™ (available from the Fanning Corp.), Cutina™ HR (available from Cognis), Simulsol™ (available from CJ Petrow), EmCon™ CO (available from Amisol Co.), Lipvol™ CO, SES, and HS-K (available from Lipo), and Sterotex™ HM (available from Abitec Corp.). Other suitable vegetable oils, including sesame, castor, corn, and cottonseed oils, include those listed in R. C. Rowe and P. J. Shesky, Handbook of pharmaceutical excipients, (2006), 5th ed., which is incorporated herein by reference in its entirety.

### EXAMPLES

### Example 1

### Dexpramipexole Binds to a Site in Complex V, Inhibiting Mitochondrial Leak Conductance and Improving Cellular Bioenergetic Efficiency

Dexpramipexole (KNS-760704; (6R)-4,5,6,7-tetrahydro-N-6-propyl-2,6-benzothiazole-diamine) is a neuroprotective drug showing promising early clinical results in amyotrophic lateral sclerosis (ALS). Dexpramipexole inhibited proteasome inhibitor- or calcium-induced ion conductance in rat brain-derived mitochondria, as did the mitochondrial permeability transition pore (mPTP) blocker cyclosporine A (CSA). Dexpramipexole and CSA also inhibited a novel ATP-sensitive conductance recorded from sub-mitochondrial vesicles (SMVs) enriched in F1F_{O} ATP synthase. Dexpramipexole failed to elicit ion conductance changes in SMVs lacking functional F1, and ¹⁴C-dexpramipexole bound specifically to purified recombinant b and OSCP subunits of the F1F_{O} ATP synthase. Dexpramipexole maintained or increased ATP levels in neurons while oxygen consumption was decreased, indicating an increase in bioenergetic efficiency, and dexpramipexole normalized the metabolic profile of proteasome-inhibitor treated cells. Dexpramipexole may act to increase the efficiency of oxidative phosphorylation in neurons at risk by inhibiting a metabolic ‘leak’ conductance associated with complex V.

ALS is a rapidly progressive, fatal neuromuscular disease characterized by the loss of upper and lower motor neurons. Mitochondrial dysfunction has been implicated in ALS, including changes in the structural integrity of mitochondria, disruption of energy metabolism and abnormal calcium (Ca²⁺) buffering. Mitochondrial dysfunction may be common to other chronic neurodegenerative disorders (NDDs), suggesting that when critical proportions of mitochondria in specific neuronal populations become dysfunctional, neurons are put at risk. Critical functions are compromised and cell death may result if ATP synthesis cannot match cellular energy requirements. The present study examined the mitochondrial actions of the candidate ALS drug dexpramipexole (KNS-760704; (6R)-4,5,6,7-tetrahydro-N-6-propyl-2,6-benzothiazole-diamine). Evidence is presented that shows a novel enhancement of the efficiency of oxidative phosphorylation by dexpramipexole resulting from the inhibition of a ‘leak’ conductance associated with the F1F_{O} ATP synthase. Dexpramipexole is a neuroprotective drug previously suggested to slow ALS disease progression in an open label clinical trial. In a recent Phase 1 clinical trial it was shown to be safe and well-tolerated at doses that should produce pharmacodynamically effective concentrations in the CNS. Dexpramipexole also demonstrated unprecedented trends in efficacy, including reduction of functional decline and decreased mortality in a double-blind, placebo- and low-dose-controlled 2-part Phase 2 study in subjects with ALS (Cudkowicz et al., in press).

Dexpramipexole is the non-dopaminergic R(+) enantiomer of the dopamine agonist and Parkinson's disease therapeutic pramipexole (Mirapex®; (6S)-4,5,6,7-tetrahydro-N6-propyl-2,6-benzothiazole-diamine). Pramipexole is protective by a non-dopaminergic mechanism in in vitro and in vivo models of cell death and NDDs, but only at higher concentrations (≧10 μM) than would be tolerated in humans. Dexpramipexole is equally protective and is tolerated at clinical doses which allow its use as a neuroprotective drug. Both enantiomers were believed to act at the level of the mitochondrion, but their specific mechanism of action remained elusive.

Mitochondria produce adenosine triphosphate (ATP) by oxidative phosphorylation, and the efficiency of this process can be affected by membrane currents that uncouple the electron transport system and oxidative phosphorylation. Mitochondrial membrane conductances participate in the initiation of cell death but also play a major role in controlling the daily metabolic health of cells. Ion channels in dysfunctional neuronal mitochondria play a direct role in the onset of cell death in conditions such as neuronal trauma and hypoxia/ischemia, but may function differently in chronic NDDs such as ALS. Recently, pramipexole was shown to inhibit calcium (Ca2+)-induced membrane currents in rat liver mitoplasts. In the current study it was found that dexpramipexole inhibited stress-induced membrane currents in brain-derived mitochondria, also inhibited similar currents in submitochondrial vesicles (SMVs), interacted with purified recombinant subunits of the F1FO ATPsynthase (complex V) while maintaining or enhancing complex V enzymatic activity. Dexpramipexole did not inhibit optically-recorded calcium-induced permeability transition in liver mitochondria, but enhanced bioenergetic efficiency in neurons and other cells. These mitochondrial effects are novel, and may underlie the cellular protective effects of dexpramipexole.

PSI Pre-Treatment in Rats Produced Currents in Isolated Brain Mitochondria that were Inhibited by Dexpramipexole and CSA

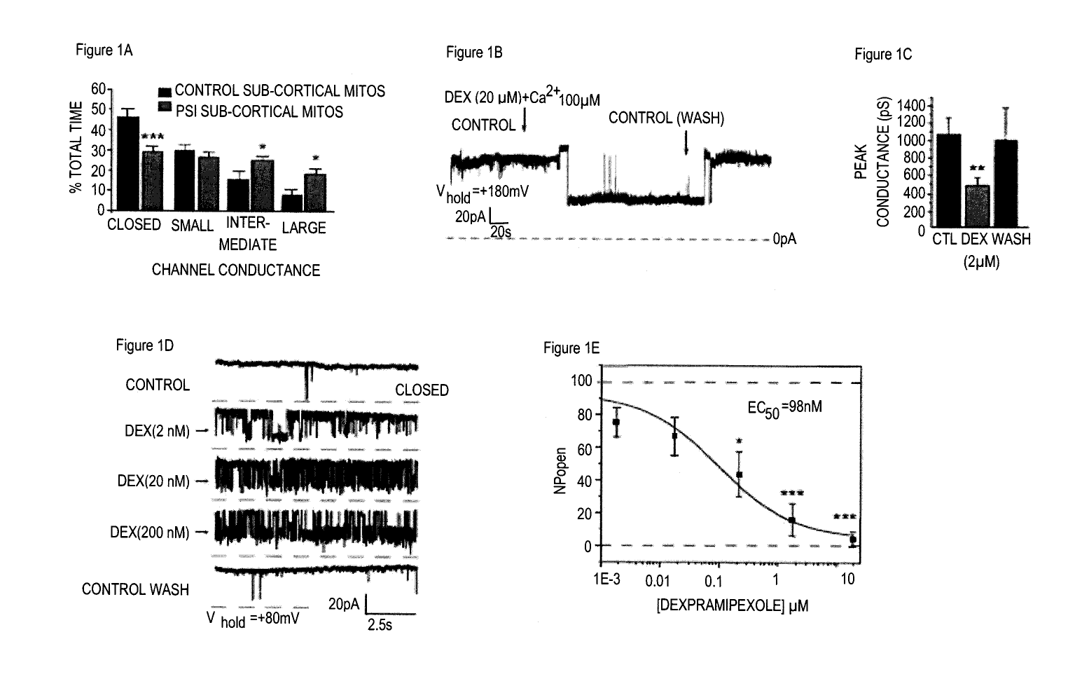

Rats were exposed in vivo to a proteasome inhibitor (carbobenzoxyl-Ile-Glu(O-t-butyl)-Ala-leucinal; PSI) to model changes in aberrant protein accumulation that may lead to chronic mitochondrial dysfunction. Mitochondria were isolated from a subcortical brain fraction after repeated dosing of rats with PSI (PSI-mitochondria, see Methods). PSI-mitochondria, incubated in the absence of added Ca²⁺, manifested intermediate- and large-conductance activity at a significantly higher frequency than mitochondria from control rat brains (FIG. 1a), and similar to the activity observed in mitochondria exposed to Ca²⁺ (as described below) or previously observed in post-ischemic brain-derived mitochondria

Dexpramipexole inhibited intermediate- and large-conductance channel activity of PSI-mitochondria (FIG. 1b, 1c, 1d); the inhibition was concentration-dependent and reversible. The decrease in the mean open probability (NP_{O}) of discrete single channel ion currents had an estimated EC₅₀ of 98 nM (FIG. 1e). Full effect of the drug could take several minutes, and washout was slow, usually requiring minutes. The concentration-response curve (CRC) was shallow, with a Hill slope <1; full inhibition of these aberrant currents (EC₁₀₀) required dexpramipexole concentrations >10 μM.

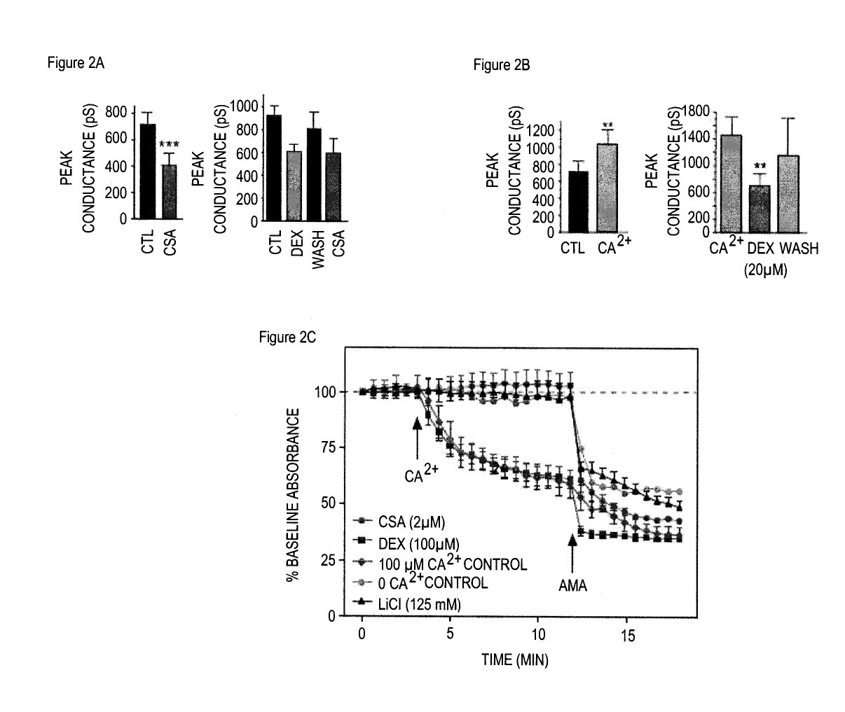

Similar to pramipexole, dexpramipexole may inhibit the pore-forming protein complex known as the mitochondrial permeability transition pore (mPTP). The mPTP participates in the initiation of some forms of cell death, although its roles in long term changes in mitochondrial function are likely to be different from its roles in more acute phenomena. Addition of Ca²⁺ to isolated mitochondria activates the mPTP and cyclosporine A (CSA) specifically inhibits it. CSA effectively blocked channel activity in PSI-mitochondria and significantly decreased the peak membrane conductance (FIG. 2a). Current that was sensitive to dexpramipexole was equally sensitive to CSA after dexpramipexole washout. Dexpramipexole-sensitive currents in PSI-mitochondria therefore share features with the mPTP, such as inhibition by CSA.

Dexpramipexole Also Inhibited Outer Membrane Ion Channel Conductances in [Ca²⁺]-Treated Brain-Derived Mitochondria

To determine if dexpramipexole also modulates mitochondrial ion channel activity during Ca²⁺-induced mitochondrial stress, isolated control rat brain mitochondria were challenged with high [Ca²⁺], resulting in an increase in intermediate- (200-750 pS) and large-conductance (>750 pS) currents recorded under whole-organelle patch clamp. Addition of 100 μM Ca²⁺ resulted in an increase in conductance (FIG. 2b), and dexpramipexole (2 or 20 μM, in 100 μM Ca²⁺) effectively decreased these mitochondrial membrane currents. These data demonstrate that dexpramipexole also potently inhibited Ca²⁺-induced ion conductance in mitochondria.

Dexpramipexole Did not Inhibit Ca²⁺-Induced Mitochondrial Transition in Liver-Derived Mitochondria

Unlike a previous report using pramipexole, no effect was found of dexpramipexole (or pramipexole; not shown) on permeability transition, optically recorded following addition of high (50-100 μM) external Ca²⁺ to liver mitochondria, even when the drug concentration was as high as 100 μM. In these experiments, known inhibitors of permeability transition, including CSA and lithium chloride (LiCl), were effective at blocking Ca²⁺-induced mitochondrial permeability transition (FIG. 2c). In brain mitochondria, Dexpramipexole-sensitive currents may have another role apart from permeability transition in mitochondrial function.

Patch Clamp Recordings of SMVs Containing F1F_{O} ATPase Revealed a Dexpramipexole-Sensitive Leak Conductance

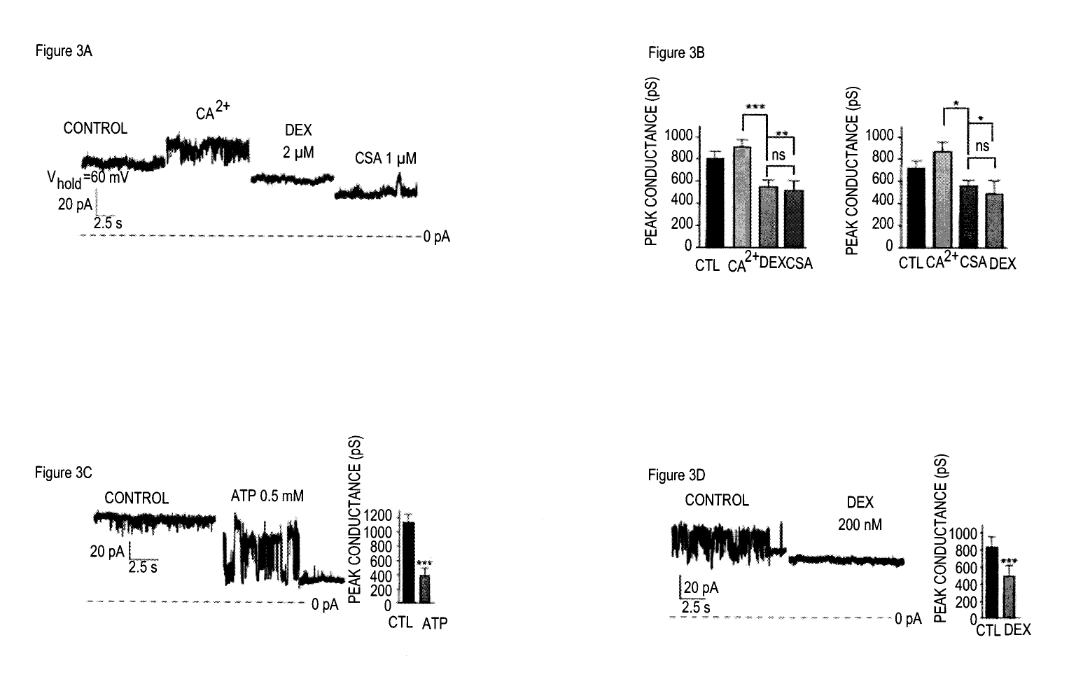

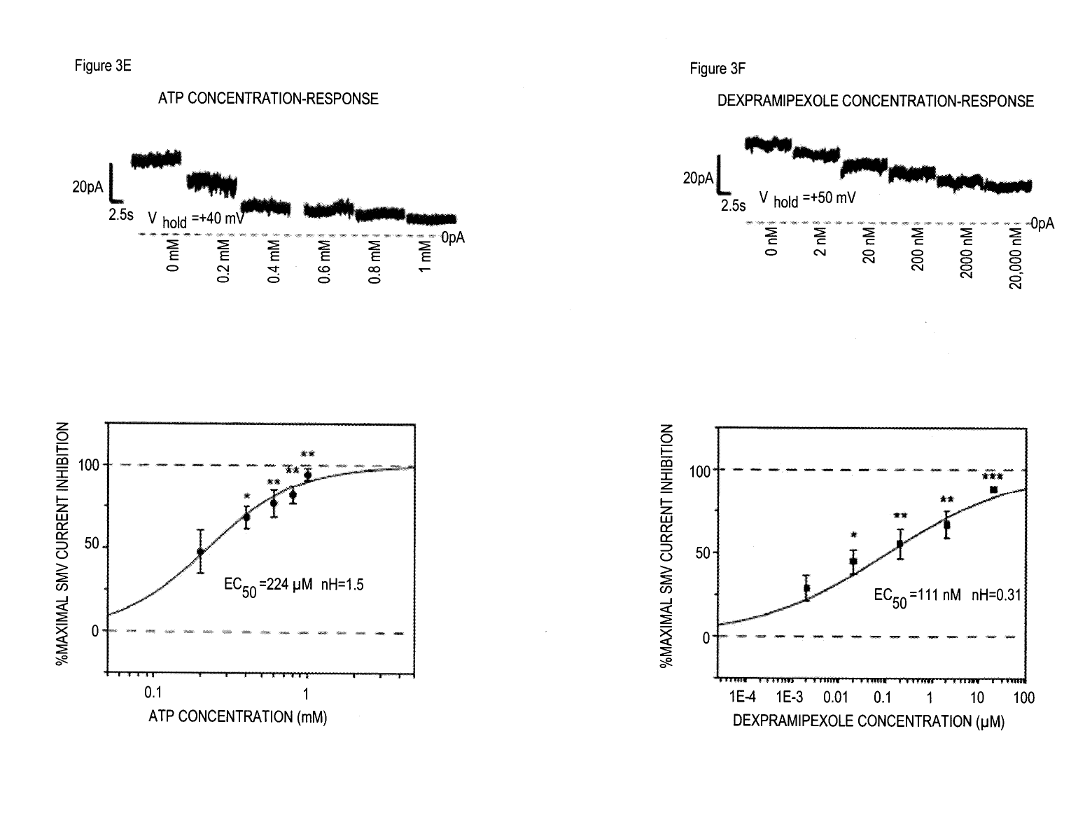

An increase in inner membrane leak conductance could decrease mitochondrial metabolic coupling, thereby reducing the efficiency of oxidative phosphorylation and putting cells at risk. To test if the dexpramipexole-inhibited conductance was expressed at the level of the inner mitochondrial membrane and might function as a ‘metabolic leak’, brain-derived SMVs enriched in F1F_{O} ATP synthase (complex V) were patch-clamped. In the absence of ATP, giga-ohm seals were formed on SMVs and high levels of conductance (peak levels 600-1200 pS), were recorded. Currents in SMVs were only modestly enhanced by high [Ca²⁺] but were inhibited by both dexpramipexole and CSA (FIG. 3a,3b). Maximal levels of inhibition by these compounds were approximately equal, and they showed only insignificant levels of additivity (FIG. 3b). The effects were also observed without added Ca²⁺, and the effects of dexpramipexole were reversible (data not shown). ATP, a known blocker of the mPTP as well as a metabolic modulator, also decreased peak membrane conductance of SMVs (FIG. 3c). The maximal effect of ATP was greater than the effect of dexpramipexole (FIG. 3c, 3d), and both ATP and dexpramipexole decreased the conductance of SMV membrane patches in a concentration-dependent manner (FIG. 3e, 3f). Dexpramipexole was potent, with an EC₅₀=111 nM, very similar to the value obtained in PSI-mitochondria, and the curve was again quite shallow, with a Hill slope <<1. ATP was much less potent (EC₅₀=224 μM), but the Hill slope was >1, and ATP was more effective, consistently blocking more of the total conductance.

Dexpramipexole Enhanced Metabolic Efficiency of Cultured Cells

To determine if the effects of dexpramipexole measured in isolated mitochondria and SMVs result in altered mitochondrial function, including changes in ATP levels and oxygen uptake in neurons, groups of cultured hippocampal neurons (DIV 14) were treated with dexpramipexole.

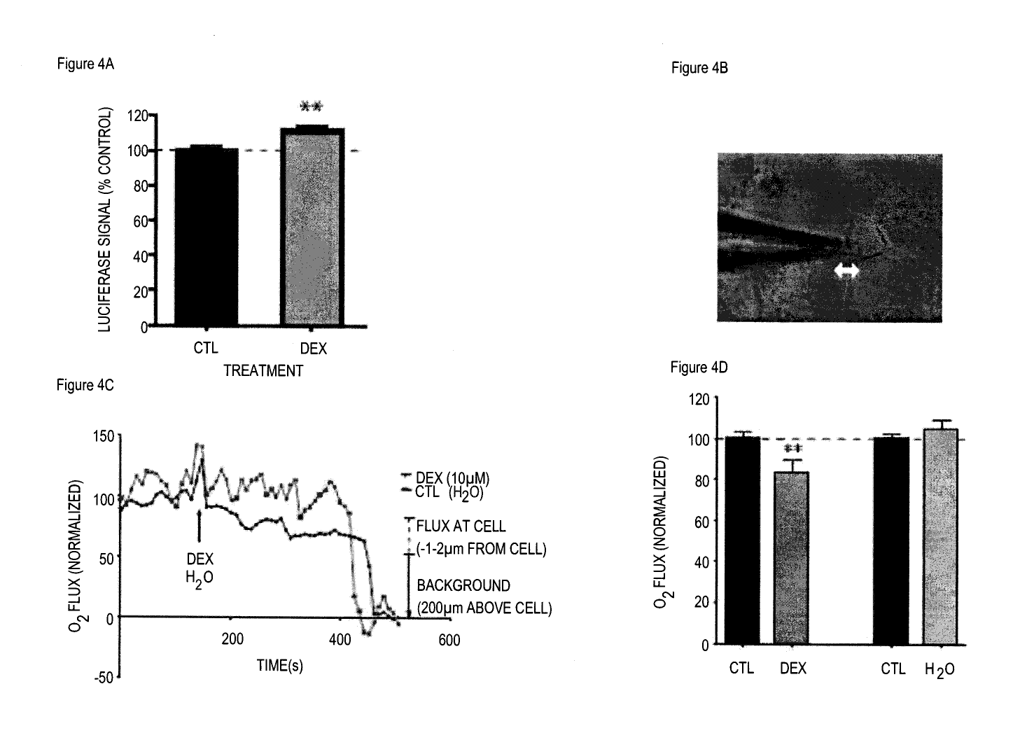

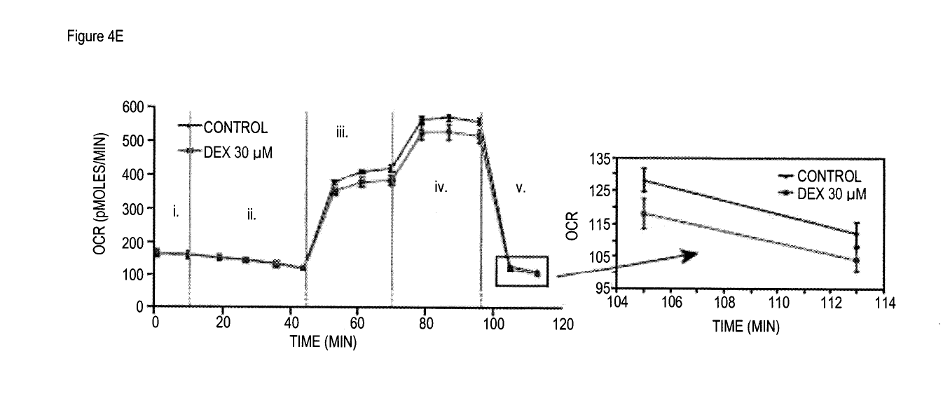

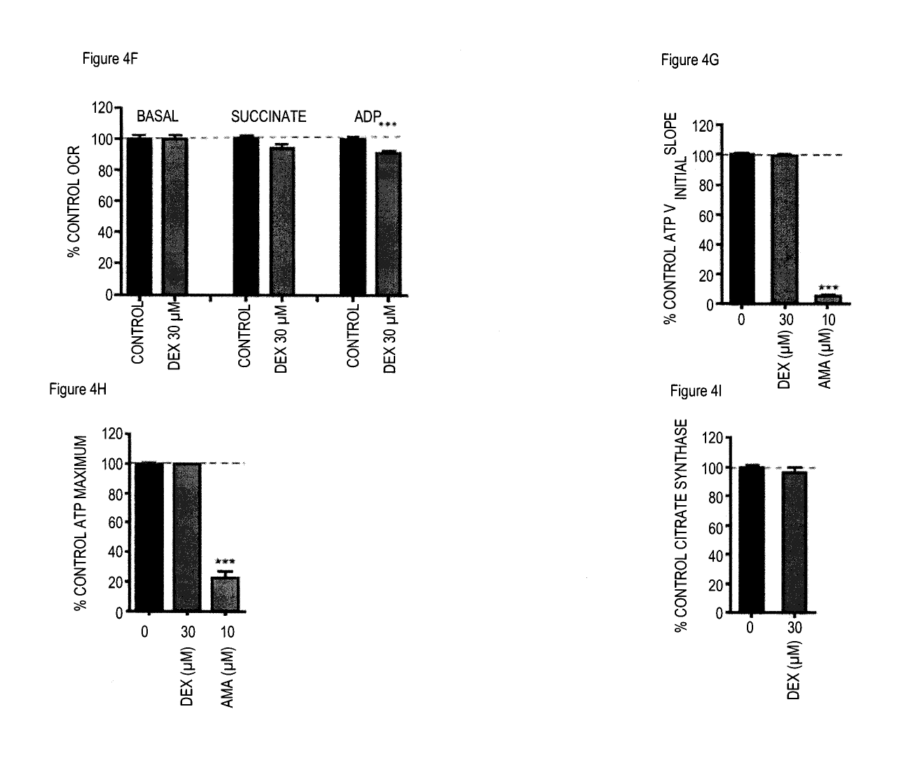

ATP levels in neurons treated with dexpramipexole (10 μM) were increased by 11% compared to vehicle-treated cultures (FIG. 4a). Under basal conditions, mitochondrial oxygen uptake contributes both to the production of ATP by oxidative phosphorylation and to counteracting ‘leak’ of protons which can decrease the coupling between the electron transport system and ATP production by reducing proton-motive force. To determine if oxygen uptake was altered by dexpramipexole, oxygen flux was measured in single cultured hippocampal neurons using a self-referencing oxygen-sensitive electrode (FIG. 4b, 4c, 4d). Single neuron measurements target the cell of interest, and self-referencing electrodes improve the signal:noise ratio compared to fluorescence-based techniques. The average oxygen flux of control neurons was stable over 5 min. Acute application of 10 μM dexpramipexole lowered average oxygen flux by ˜16% percent to a new stable value after 1-5 min (FIG. 4c,4d). These data, in conjunction with the increased ATP levels in identically-treated neurons, suggest that dexpramipexole-treated neurons couple ATP production more efficiently to oxygen uptake. Similarly, incubation of SH-SY5Y neuroblastoma cells in dexpramipexole (1-100 μM; 24 hr), significantly increased ATP levels, relative to control values, with maximal group increases ˜18%; visualized using a Seahorse® respirometry system. Dexpramipexole (30 μM) also modestly decreased basal oxygen consumption rate (OCR) in these cells. SH-SY5Y cells were also incubated in galactose, a sugar that exclusively requires mitochondrial (rather than glycolytic) metabolism to produce ATP. Cells treated with dexpramipexole in galactose-containing medium had similar dexpramipexole-induced increases in ATP levels, suggesting that the effects of dexpramipexole on cellular ATP levels were mediated via effects on mitochondrial metabolism.

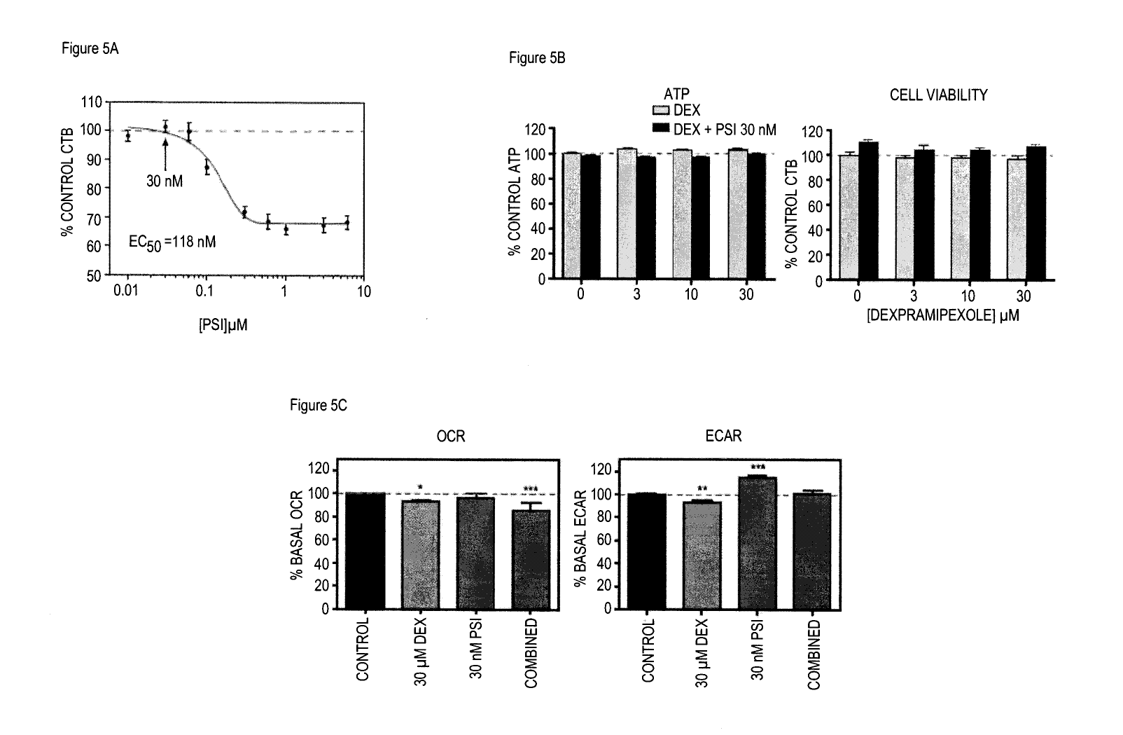

In digitonin-permeabilized cultured cortical neurons incubated in control medium containing the complex I inhibitor rotenone (FIG. 4ei), acute application of dexpramipexole (30 μM) did not affect the already low levels of basal respiration (FIG. 4eii). In control cells, addition of the complex II substrate succinate produced a significant increase in OCR, measured using a Seahorse® multi-well flux analyzer (FIG. 4eiii); following a pulse of ADP, OCR was further increased (FIG. 4eiv). In dexpramipexole-treated cells, the increase in OCR produced by succinate and following the ADP pulse was less than in control cells (FIG. 4eiii,4eiv), and was lower than control levels in the presence of the complex V inhibitor oligomycin (FIG. 4e, inset). In other experiments, when dexpramipexole was applied to digitonin-treated cortical neurons 1 hr. prior to the onset of recording, cells likewise had lower OCR during succinate incubation, and significantly lower OCR following an ADP pulse, relative to controls (FIG. 4f). In these cells measurements of the initial slope of ATP synthesis following an ADP pulse, maximal ATP levels, and levels of citrate synthase, an indicator of mitochondrial number and integrity, were unchanged in dexpramipexole (FIG. 4g, 4h, 4i). The Seahorse® respirometry system was also used with cultured C2C12 myoblasts to determine the effects of PSI (18 hr incubation) on bioenergetic profiles. In C2C12 cells, a CRC for cell death by PSI was established (FIG. 5a), and a PSI concentration that was near the inflection point of the killing curve (30 nM) was used to stress but not kill C2C12 cells in subsequent experiments. PSI-treated or control C2C12 cells were incubated in dexpramipexole (30 μM) or control medium, and relative OCR and the extracellular acidification rate (ECAR) were measured. Under these conditions, neither ATP levels nor cell viability (FIG. 5b) were significantly affected in any group (PSI, dexpramipexole or the combination, relative to control). In PSI, C2C12 cells did not display a significant change in OCR, but did display a very significant increase in ECAR (FIG. 5c), indicating a higher relative level of glycolytic activity following low-level PSI treatment. In contrast, cells incubated only in dexpramipexole displayed a significant decrease in OCR, which was not accompanied by a change in ECAR (FIG. 5c), indicative of an increase in basal mitochondrial efficiency of dexpramipexole-treated cells. Co-incubation of dexpramipexole and PSI resulted in a profile identical to dexpramipexole alone, with a significant decrease in OCR and no increase in ECAR (FIG. 5c). This normalization of respiratory profiles may be protective. When SH-SY5Y cells were exposed to levels of PSI that produced cell death, pretreatment with dexpramipexole at concentrations producing effects on bioenergetic efficiency were protective.

Dexpramipexole Enhanced the Enzymatic Activity of Complex V

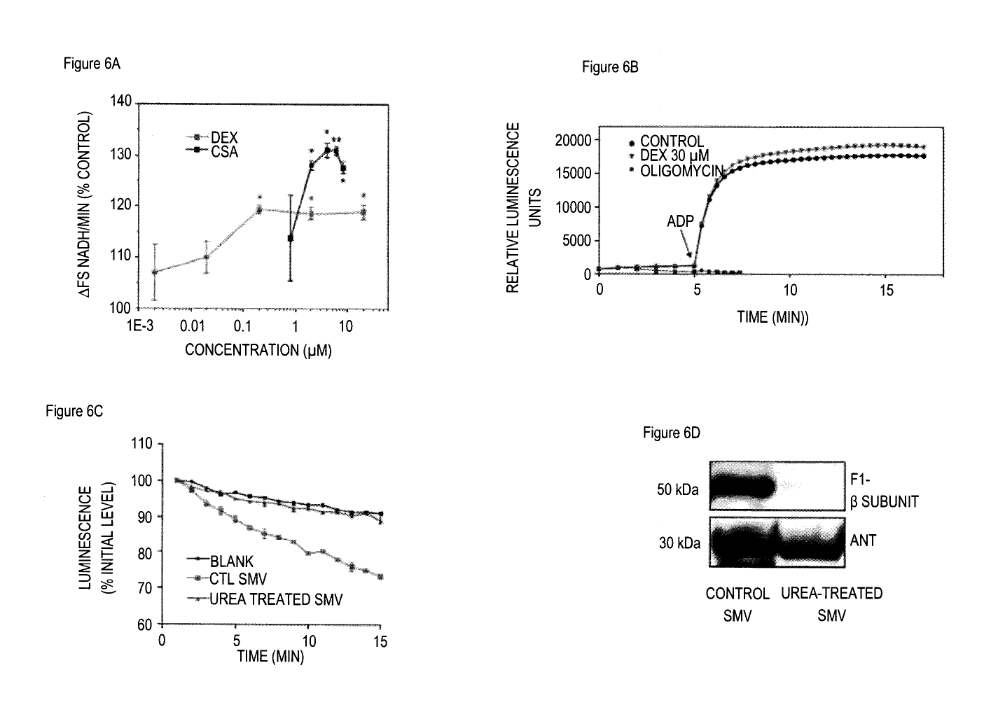

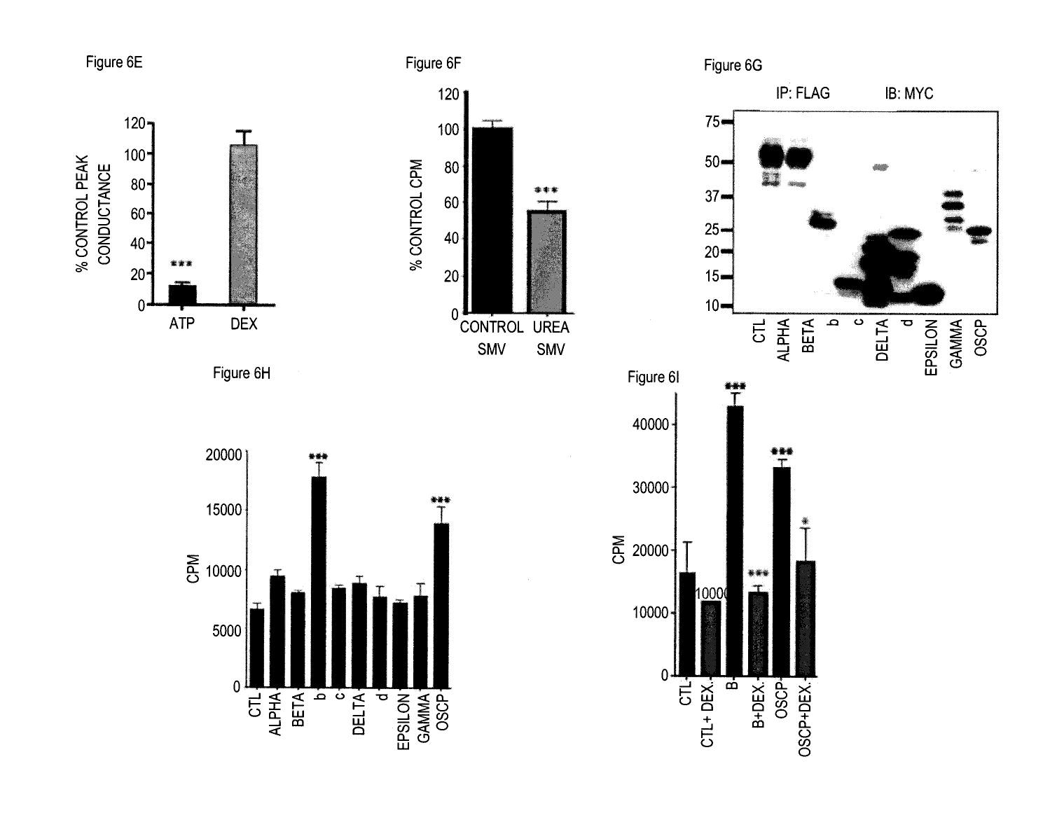

Dexpramipexole-sensitive conductance in SMVs was eliminated by removal of F1, suggesting an interaction of dexpramipexole with complex V, and suggesting that regulation of this current might occur concurrently with regulation of ATP synthesis and hydrolysis. To determine if such interaction resulted in alteration of the enzymatic function of complex V, 3 different techniques were employed to measure the ability of complex V to hydrolyze or synthesize ATP in the presence and absence of dexpramipexole. SMVs can hydrolyze ATP, and it was found that dexpramipexole significantly enhanced ATP hydrolysis in SMVs in a concentration-dependent manner in an assay where complex V enzymatic activity was estimated by the change in NADH signal (FIG. 6a); CSA also significantly enhanced ATP hydrolysis (FIG. 6a). In a different assay in SMVs, dexpramipexole increased ATP hydrolysis, measured by ATP-luciferase levels in the medium, with an EC₅₀=1.63 μM.

The ATP synthase inhibitor oligomycin effectively inhibited ATP hydrolysis in these assays. In isolated liver mitochondria, dexpramipexole modestly increased ATP synthesis in response to an ADP pulse (FIG. 6b). While this effect was small in these functional mitochondria, the effect of dexpramipexole in these mitochondria was significant and potent (EC₅₀=166 nM) and, like other measures of dexpramipexole's action, the CRC had a Hill slope <1 (not shown).

Dexpramipexole Binding and Current Inhibition were Reduced by Removal of F1 from the F1F_{O} Mitochondrial ATP Synthase in SMVs

Complex V has an inside-out orientation in SMV membranes, with F1 on the outside. This allowed us to examine the effects of urea treatment (see Methods), which removed/denatured non-membrane-residing components. Urea-treated SMVs were unable to perform ATP hydrolysis, and removal of F1 β subunit was confirmed by the Western blot immunoanalysis whereas the known membrane-inserted component ANT was unaffected (FIG. 6c,d). These SMVs still displayed leak conductance of similar magnitude under patch clamp (peak conductance 600-1200 pS), but dexpramipexole no longer inhibited the conductance, suggesting that the presence of a non-membrane-inserted component was critical for dexpramipexole's effects (FIG. 6e). ATP, however, still very effectively decreased the leak conductance in the absence of F1 (FIG. 6e). Finally, total binding of ¹⁴C-dexpramipexole to SMVs was determined before and after treatment with urea. In 3 independent experiments the total binding of ¹⁴C-dexpramipexole was significantly reduced (mean reduction 42%) with elimination of functional F1 (FIG. 6f).

Interaction of ¹⁴C-Dexpramipexole with Specific Subunits of Complex V

Individual subunits of complex V were heterologously expressed in 293T cells (FIG. 6g) and used to directly test for the specific binding of ¹⁴C-dexpramipexole. When incubated in ¹⁴C-dexpramipexole, two subunits, b and the oligomycin-sensitivity conferring protein (OSCP) had levels of binding that were significantly above untransfected control levels (FIG. 6h). Co-incubation in ‘cold’ dexpramipexole (100 μM) significantly reduced binding in these subunits to levels indistinguishable from untransfected controls, suggesting specific binding to the stator of the ATP synthase (FIG. 6i).

Understanding mechanisms by which dexpramipexole protects cells at risk, including neurons, is potentially important in light of the promising trends in efficacy seen in ALS patients (reference in press*). In the current study, dexpramipexole potently and effectively inhibited ion channel activity evoked by Ca²⁺ or PSI pre-treatment in brain-derived mitochondria, but, interestingly, did not block permeability transition recorded in liver mitochondria, as had been suggested previously for pramipexole. Inhibition of currents in PSI-mitochondria by dexpramipexole was very potent, but the slope of the resulting CRC was quite shallow. Potent but shallow CRC slopes characterized many of the effects of dexpramipexole observed in this study, possibly indicating negative cooperativity among multiple binding sites.

It has been shown that intermediate- and large-conductance channel activity was present at higher frequency in mitochondria isolated from affected brain regions of rodents exposed in vivo to global ischemic injury. These currents were inhibited by divalent chelation, by immunological inhibition of BCL-xL and by inhibition of VDAC. This suggested that a mitochondrial channel comprised of a complex of proteins was present after ischemic injury, made up of components of the mitochondrial permeability transition pore (mPTP) at the inner mitochondrial membrane, as well as a pro-apoptotic version of BCL-xL (ΔN BCL-xL) and VDAC in the outer membrane. In the current study similar mitochondrial currents were induced in brain-derived mitochondria by Ca²⁺ and PSI; these currents were inhibited by dexpramipexole and by the mPTP inhibitor CSA, suggesting that aberrant conductances recorded in the two models share some biophysical, and possibly, pharmacological features. Patch-clamp recordings of SMVs also revealed substantial dexpramipexole-, CSA- and ATP-sensitive conductance in the absence of challenge with Ca²⁺ or treatment with PSI, possibly due to preparation of the SMVs themselves.

Leak conductance associated with the mitochondrial inner membrane could lead to shunting of the proton-motive force that provides the energy for oxidative phosphorylation, with consequences for bioenergetic efficiency. Dexpramipexole application resulted in modest but potent enhancement of ATP synthesis in liver-derived mitochondria and a less-potent but significant increase in ATP hydrolytic potential in SMVs. Without being bound by any theory, it believed that the two phenomena, enzymatic modulation and current inhibition by dexpramipexole, are related and result from an interaction with F1 of complex V, and lead to an increase in the efficiency of oxidative phosphorylation. Increased ATP levels were observed in cultured neurons and other cells after exposure to cytoprotective (≧10 μM) concentrations of dexpramipexole, accompanied by lower basal OCR. Incubation in dexpramipexole maintained ATP levels in stressed cells and decreased succinate- and ADP-stimulated OCR in rotenone-treated cultured cortical neurons. In addition, dexpramipexole normalized ECAR levels and lowered OCR in PSI-treated C2C12 cells, suggesting that it ameliorated PSI-induced mitochondrial stress.

Removal of F1 resulted in a loss of hydrolytic activity of complex V, loss of dexpramipexole- but not ATP-mediated inhibition of conductance, and significantly reduced binding of radiolabeled dexpramipexole. The very similar potencies of dexpramipexole in brain-derived PSI-mitochondria and SMVs, and the inhibition of both conductances by CSA, suggested that the currents in the different preparations are comprised of common components, and have features in common with the mPTP. Further examination of the binding of radiolabeled dexpramipexole to complex V, using individual subunits isolated following heterologous expression in 293T cells demonstrated that the apparent target(s) of dexpramipexole binding are in fact within the non-membrane-inserted portion of the ATP synthase in subunits comprising the closely associated ‘stator’ stalk external to F1. This structure is involved in stabilizing F1 so that it can perform its rotary functions in the synthesis of ATP (REF), but unlike previous ligands that have been shown to interact with the OSCP, dexpramipexole did not inhibit the enzymatic activity of the complex. This is novel, and the mechanism of such an interaction at this point would be speculative.

Dexpramipexole inhibited PSI-induced cell death in a concentration-dependent manner when tested in SH-SY5Y neuroblastoma cells. In this study, effective cytoprotective concentrations of dexpramipexole (or pramipexole), and concentrations required for significant effects on metabolic efficiency in cells, were significantly higher than the EC₅₀s for dexpramipexole-induced mitochondrial current inhibition or enhancement of ATP synthesis. Because of the shallow slopes of the CRCs, concentrations producing complete elimination of these aberrant currents (an EC₁₀₀), and corresponding to a 10-30 μM level may be required for enhanced bioenergetic efficiency and significant cytoprotection.

Mitochondrial dysfunction has long been implicated in the pathogenesis of neurodegenerative disease. Proteasomal dysfunction is a potential mechanism for accumulation of undegraded proteins in mitochondria, and this has been suggested as one of many potentially stressful events coupled to the onset of neurodegeneration in Alzheimer's disease, ALS and PD. It has been demonstrated that mitochondrial stress induced by PSI (or Ca²⁺) results in increased membrane current in mitochondrial membranes, conductance that is expressed at high levels in otherwise untreated SMVs. This membrane current constitutes a leak conductance, probably closely associated with complex V, that results in a reduction in bioenergetic efficiency. If this inefficiency cannot be countered by sufficient compensatory increases in available substrate metabolism and OCR, or by shifting to glycolysis, it may stress energy supplies, resulting in increased risk of neuronal death when energy demand exceeds supply. These data suggest that dexpramipexole reduces this risk by inhibiting aberrant complex V-associated leak conductance, resulting in neuroprotection and, quite possibly, therapeutic benefit in NDDs. Further, these data suggest that an important substrate for the regulation of the efficiency of oxidative phosphorylation resides within the subunit structure of complex V itself.

FIG. 1 shows that dexpramipexole inhibited PSI-induced currents in brain-derived mitochondria. The panels of FIG. 1 are described as the following: FIG. 1a. Repeated pre-treatment of rats with PSI produced a significant increase in intermediate- and large-conductance mitochondrial ion channel activity of mitochondria isolated from subcortex, relative to mitochondria obtained from control animals. Histograms show the % total time that patches displayed specified conductance levels in all recorded traces (Closed, small<200 pS, intermediate>200 pS<750 pS, large>750 pS; n=190 recordings from a total of 10 PSI-exposed sub-cortical mitochondria, n=83 recordings from a total of 9 vehicle (DMSO)-exposed sub-cortical mitochondria). PSI in this case was dosed every other day for 1 week; animals were sacrificed and mitochondria were isolated at the end of PSI dosing. Conductance level frequencies for PSI-dosed mitochondria were only compared with the comparable level in non-PSI-dosed mitochondria, using 2-tailed unpaired t-tests; p=0.0005 for closed PSI-mitochondria compared to closed control, p=0.0426 for intermediate conductance PSI-mitochondria compared to intermediate control, p=0.0192 for large PSI-mitochondria compared to large control. In all figures, the specific analyses performed and unadjusted or adjusted p values connoting significance are presented. The general level of significance obtained when comparing 2 groups, including pre-planned post hoc comparisons, is indicated on any figure by the number of asterisks above or below the mean value; *=p<0.05, **=p<0.01, ***=p<0.0001.

FIG. 1b. Example of a continuous patch clamp recording from a PSI-mitochondrion (see Methods) in normal recording medium, and the reversible reduction in leak conductance during the bath application of dexpramipexole (DEX). FIG. 1c. Group data showing that dexpramipexole (2 μM) significantly and reversibly decreased peak conductance recorded from mitochondria isolated from PSI-injected rat subcortex (n=15 mitochondria, except for wash, where n=6 mitochondria). 2-tailed paired t-test, p=0.0352. The wash was not included in the analysis. FIG. 1d. Dexpramipexole produced a concentration-dependent decrease in the mean open probability of intermediate-conductance (˜500 pS) channels recorded from PSI mitochondria. This example shows recordings from the same organelle-attached patch made in control medium, 2, 20 and 200 nM dexpramipexole, and during a wash to control medium; holding potential of +80 mV. Note that in this example many closures reveal sub-conductance states. Sample recordings were obtained at steady-state for each condition.

FIG. 1e. Concentration-response relationship showing the mean inhibitory effect of different concentrations of dexpramipexole on NP_{O} (number of channels multiplied by the probability of opening of each channel), recorded in brain-derived PSI-mitochondria (n=5 mitochondria for all except 20 μM, where n=9). One-way ANOVA, p=0.000014; pre-planned post hoc comparisons, Bonferroni corrected t-tests, p=0.0455 for 200 nM dexpramipexole, p=0.00038 for 2 μM dexpramipexole, p=0.0005 for 20 μM dexpramipexole; EC₅₀=98 nM by logistic fit; Hill slope, nH<1.

FIG. 2 shows that cyclosporine A (CSA) reduced peak conductance in dexpramipexole-sensitive PSI-mitochondria, and high calcium (Ca²⁺) induced dexpramipexole-sensitive currents in normal brain mitochondria. FIG. 2 also shows that dexpramipexole did not inhibit mitochondrial permeability transition recorded in rat liver mitochondria. FIG. 2a. (Left) Bar graph showing the mean maximal level of inhibition of peak conductance by 1 μM CSA in recordings from PSI-mitochondria (n=7 mitochondria); p=0.0003, paired t-test. (Right) Bar graph showing the mean maximal inhibition of peak conductance by 200 nM dexpramipexole in PSI-mitochondria, followed by partial recovery in a control medium wash (>5 min.), followed by maximal inhibition by 1.0 μM CSA (n=3 mitochondria). FIG. 2b. (Left) Bar chart showing the mean effect of 100 μM Ca²⁺ on peak membrane conductance (in pS) relative to control medium. (n=14 mitochondria); p=0.0092, 2-tailed paired t-test. (Right) Bar chart showing the mean inhibition of peak conductance (in pS) by 20 μM dexpramipexole in the continued presence of Ca²⁺ (n=7 mitochondria); p=0.0094, 2-tailed paired t-test; n=4 for wash; the wash was not included in the analyses. FIG. 2c. Baseline optical absorbance of fresh, respiring rat liver mitochondria (measured respiratory control ratio>5) in response to 100 μM Ca²⁺ or alamethicin (AM) exemplifying the phenomenon of mitochondrial permeability transition in the presence of the indicated agents. Each point represents the mean±SEM for the group at each time point; n≧12 wells for each condition. Dexpramipexole, unlike CSA, did not inhibit mitochondrial transition in liver mitochondria.

FIG. 3 shows that dexpramipexole and CSA decreased conductance of submitochondrial vesicles (SMVs). FIG. 3a shows an example of a patch clamp recording from a brain-derived SMV; holding potential +60 mV, before and after addition of the indicated agents to the bath (CSA is 1 μM). FIG. 3b. shows histograms represent group data (mean±SEM) depicting effects of the indicated compounds on peak conductance of SMV patches. In all cases current was measured from 0 pA and is presented as peak conductance assuming a linear current-voltage relationship. (Left) Level of conductance before and after switching to high [Ca²⁺], Ca²⁺ plus 2 μM dexpramipexole (n=13 SMVs), followed by Ca²⁺, dexpramipexole plus 1.0 μM CSA (n=5 SMVs). One-way ANOVA, p=0.0002; pre-planned post hoc Bonferroni corrected t-tests, Ca²⁺ vs. 2 μM dexpramipexole, p=0.00041, Ca²⁺ vs. CSA, p=0.0048. (Right) Level of conductance in SMVs in control medium, after switching to high [Ca²⁺], followed by Ca²⁺ plus 1.0 μM CSA (n=5 SMVs), followed by Ca²⁺, CSA and 2 μM dexpramipexole (n=3 SMVs). One-way ANOVA, p=0.0043; pre-planned post hoc Bonferroni corrected t-tests, Ca²⁺ vs. CSA, p=0.0196, Ca²⁺ vs. dexpramipexole, p=0.046. FIG. 3c shows an example of an SMV recording before and after addition of 0.5 mM ATP; holding voltage+110 mV; discontinuous recording, ATP was added <1 min. prior to the break in the recording; Bar chart indicates group data (mean±SEM) for the effect of 0.5 mM ATP on peak conductance level (n=9 SMVs; p=0.0001, paired t-test).

FIG. 3d shows an example of an SMV recording before and after addition of 200 nM dexpramipexole; discontinuous recording, addition of dexpramipexole occurred prior to resumption of recording; holding potential +100 mV. Histograms indicate group data for the effect of 200 nM dexpramipexole on peak conductance level (n=6 SMVs; p=0.0003, paired t-test). FIG. 3e shows an ATP decreased peak conductance levels in a concentration-dependent manner. Example shows recordings from a single SMV patch, indicating steady-state changes in conductance level at the indicated concentrations of ATP; holding potential +40 mV. Group data in the box below shows the logistic fit of the effect of ATP concentration on % maximal SMV current inhibition (n=4 SMVs; EC₅₀=224 μM; nH=1.5). One-way ANOVA, p=0.0001; pre-selected post hoc Bonferroni corrected t-tests, control vs. 0.4 μM, p=0.010, 0.6 μM, p=0.0032, 0.8 μM, p=0.0023, 1.0 μM, p=0.0016. FIG. 3f. shows dexpramipexole decreased peak conductance levels in SMVs in a concentration-dependent manner. As in FIG. 3e., the example shows recordings from a single SMV patch, indicating steady-state changes in conductance level at the indicated concentrations of dexpramipexole; holding potential +50 mV. Note that dexpramipexole inhibits a smaller percentage of peak total current than ATP, which was a general finding in this preparation. Group data below the traces shows the logistic fit of the mean (±SEM) % maximal current inhibition at different concentrations of dexpramipexole (n=5 SMVs; EC50=111 nM; nH=0.31). One-way ANOVA, p<0.0001; pre-selected post hoc Bonferroni corrected t-tests, control vs. 20 nM dexpramipexole, p=0.0375, 200 nM, p=0.009, 2 μM, p=0.0025, 20 μM, p=0.0005.

FIG. 4 shows the dexpramipexole modulation of cellular bioenergetics. FIG. 4a. shows dexpramipexole (10 μM, 12 hr. pre-treatment) significantly increased ATP levels, measured in a luciferase assay (ATP-Glo kit) in cultured hippocampal neurons (n=21 wells each condition, 2 independent cultures, p=0.0024, unpaired t-test). FIG. 4b. shows an image of an oxygen-sensitive electrode placed in position to record oxygen uptake by a single cultured hippocampal neuron. Shown are the recording and self-referencing positions of the electrode. Scale bar with arrow represents 10 μm; measurement of oxygen level in proximity of neurons occurred ˜1-2 μm from cell surface and the reference point was 10-12 μm away from cell surface. FIG. 4c. shows an example of a single self-referencing oxygen electrode recording from a cultured hippocampal neuron after addition of dexpramipexole (10 μM) or an equivalent volume of water to the bath.

FIG. 4d. shows a histogram showing group data (mean±SEM) for oxygen flux measured by the self-referencing oxygen electrode system from individual neurons before and after addition of dexpramipexole (10 μM; n=14 neurons) or an equivalent volume of water (n=6 neurons). Unpaired t-test, p<0.0001. FIG. 4e. shows dexpramipexole treatment decreased OCR following both succinate and ADP injection. Primary rat cortical cultures were incubated with digitonin (10 μg/ml) and rotenone (100 nM) for 45 minutes in MAS1 buffer prior to measurement of oxygen consumption rate (OCR) by a Seahorse® flux analyzer. OCR at baseline (i.), 30 μM dexpramipexole (n=10 wells) or vehicle injection (n=10 wells) (ii.), 10 mM succinate (iii.), 1 mM ADP (iv.) and 10 μM antimycin A (v.). FIG. 4f. shows dexpramipexole treatment reduced OCR, relative to corresponding control values, following succinate (p=0.1211; 2-tailed t-test) and ADP injection (p=0.0001; 2-tailed t-test). Primary rat cortical cultures were pretreated with dexpramipexole (30 μM; n=95 wells) or control medium (n=94 wells) for 1 hr., then incubated with digitonin (10 μg/ml), rotenone (100 nM), and dexpramipexole (30 μM) prior to OCR measurements. Data are shown as a percentage of the control group OCR±SEM. In cortical neurons, dexpramipexole treatment at 30 μM as in FIG. 4f. did not affect the initial slope (FIG. 4g.) or maximal level (FIG. 4h.) of ATP production (see Methods) following ADP addition, antimycin A (AMA) significantly reduced the initial slope and maximal levels of ATP production (One-way ANOVA, Bonferroni-corrected pre-planned post hoc t-tests; p<<0.001). FIG. 4i. shos that dexpramipexole treatment did not significantly affect citrate synthase levels.

FIG. 5 shows that dexpramipexole altered respiration parameters and ATP production in the C2C12 myoblast cell line. FIG. 10. a. shows PSI reduced cellular viability of C2C12 cells after 18 hr. exposure at concentrations >60 nM as determined by CellTiter-Blue (CTB) assay. Data are presented as a percentage of the vehicle-treated control ±SEM (n=10 wells at each concentration). A sub-lethal concentration of PSI, 30 nM, was used in subsequent experiments to stress C2C12 cells. FIG. 10. b. shows that cells compensate for PSI-induced stress, and ATP levels and viability are maintained over the experimental time course. C212 cells were grown for 24 hr. after plating into 96 well plates and exposed to either dexpramipexole (at indicated concentrations) and/or PSI (30 nM), or neither drug (control). Experiments commenced 18 hr. after addition of drug. (Left) ATP levels measured using a static luciferin-luciferase assay (ATP-Glo kit) (2-way-ANOVA; p=0.1763; n=18 wells for each group) or (Right) cell viability determined using CellTiter-Blue (2-way-ANOVA, p=0.2824). Data are presented as a percentage of the vehicle-treated control±SEM (n=14-17 wells for each group).

FIG. 5c. shows that Dexpramipexole (30 μM; blue bars) significantly decreased basal OCR (p=0.0477) and ECAR (p=0.0002) in C212 cells. (Left) Oxygen consumption rate (OCR) and (Right) extracellular acidification rate (ECAR) of cells exposed to dexpramipexole (30 μM) and/or PSI (30 nM), or no drug (control) for 18 hr. (control, n=27; dexpramipexole 30 μM, n=24; PSI, n=12, ECAR p=0.00015; combination, n=9, OCAR p=0.0009). Data are expressed as a percentage of the corresponding control OCR or ECAR value, and p values represent results of pre-planned post hoc comparisons (Tukey HSD) following one-way ANOVA (p=0.001). Data shown represent mean±SEM. FIG. 5d. shows 24 hr. exposure of undifferentiated SH-SY5Y cells to PSI at 150 or 650 nM resulted in significant decreases in cell viability (2-factor MANOVA, dexpramipexole vs. 150 nM PSI, p=0.001, dexpramipexole vs. 650 nM PSI, p=0.001; pre-planned post hoc Bonferroni-corrected t-tests, 150 nM PSI vs. 150 nM PSI+30 μM Dex, p=0.031; 150 nM PSI+100 μM Dex, p=0.001; 650 nM PSI+100 μM Dex, p=0.001; all other comparisons were not statistically significant). Bar graphs represent normalized mean±SEM. Cell viability was measured by CellTiter-Blue®; n=41-98 wells/condition.

FIG. 6 shows modulation of complex V activity by dexpramipexole (panels a. and b.). FIG. 6a. F1F_{O} ATPase activity (ATP hydrolysis) in the presence of different concentrations of dexpramipexole (DEX; red filled squares) or CSA (black filled squares) shown as a function of the rate of decrease in NADH fluorescence and expressed as % of control (see Methods; dexpramipexole, n=3 determinations/point, one-way ANOVA, p=0.0014; CSA, n=3 determinations/point, one-way ANOVA, p=0.0002. Pre-selected Bonferroni corrected post hoc comparisons; control vs. 200 nM dexpramipexole, p=0.0315, 2 μM, p=0.0456, 20 μM, p=0.0438; control vs. 2 μM CSA, p=0.0132, 4 μM, p=0.010, 6 p=0.0096, 8 μM, p=0.0152.

FIG. 6b. Dexpramipexole (30 μM) increased ATP levels in liver mitochondria (n=3 wells/point) following an ADP pulse using a dynamic luciferin-luciferase assay. All drugs added at time 0. FIG. 6c shows urea-treatment of SMVs alters enzymatic activity, pharmacology of membrane currents and radiolabeled dexpramipexole binding (panels c.). FIG. 6c also shows c. decrease in oxyluciferin luminescence levels indicating time-dependent decreases in ATP levels in the absence (Blank) and presence (CTL SMV) of SMVs, and in the presence of urea-treated SMVs (n=3 wells for each condition). FIG. 6d. shows an immunoblot with an antibody against the F1 β subunit in protein from control SMVs or urea-treated SMVs. Bottom band shows immunoblot with an antibody against adenine nucleotide transporter (ANT) to provide a loading control. FIG. 6e. shows effects of ATP or dexpramipexole on SMV peak total conductance, expressed as % of control peak conductance, after presumptive chemical removal of F1 from SMVs by treatment with urea (n=7 SMVs for ATP; n=6 SMVs for dexpramipexole). Groups represent separate experiments; p=0.0028 for ATP, unpaired t-test. FIG. 6f. shows level of ¹⁴C-dexpramipexole binding in SMVs treated with urea, relative to control SMV levels (n=15 samples/condition, p≦0.0001, unpaired t-test).

FIG. 6g-i shows binding of radiolabeled dexpramipexole to individual heterologously-expressed subunits of complex V and competition by unlabeled dexpramipexole. FIG. 6g. shows Myc-Flag tagged constructs for human F1 FO ATP synthase subunits (as labeled at bottom of gel) immunoprecipitated with anti-FLAG affinity gel and immunoblotted with anti-Myc tag antibody. CTL lane represents immunoprecipitate with anti-FLAG affinity gel of cell lysate from non-transfected cells. FIG. 6h. shows counts per minute of anti-Flag affinity gel immunoprecipitates from cells exposed to 200 nM 14C-labeled dexpramipexole. One-way ANOVA, Bonferroni-corrected pre-planned post hoc comparisons; *** indicates p<0.001. FIG. 6i. shows counts per minute of anti-Flag affinity gel immunoprecipitates from cells exposed to 200 nM 14C-labeled dexpramipexole and 20 micro M unlabeled dexpramipexole. One-way ANOVA, Bonferroni-corrected pre-planned post hoc comparisons; * represents p<0.05,*** represents p<0.001; black asterisks are levels of significance for comparisons between Control and radiolabeled b and OSCP, red asterisks are levels of significance for comparisons between corresponding levels of binding of radiolabeled dexpramipexole in b or OSCP columns and their corresponding levels in the presence of excess unlabeled dexpramipexole.

### Example 2

### Methods

Isolation of Brain-derived Mitochondria. Standard techniques were adapted for isolating brain-derived mitochondria. Mitochondria were stored in isolation buffer (IB) at −80° C. For PSI-mitochondria, male Sprague-Dawley rats (6 wo, ˜200 gm, 3-6 rats/group/condition, at least 2 different preparations/experimental paradigm) were injected with the ubiquitin proteasome inhibitor Z-Ile-Glu(OtBu)-Ala-Leu-al (PSI; Peptides International Inc, Kentucky, USA; s.c. every other day for 1 or 2 weeks; 6.0 mg/kg PSI in DMSO, or with DMSO, 200 μL/rat), and mitochondria isolated and stored as above.

F₁F_{O} ATPase-Containing Submitochondrial Vesicle (SMV) Preparation.

Preparation of SMVs adapted from earlier methods. The lubrol-insoluble fraction (SMVs) prepared by re-suspension in IB (approx. 4-10 mg/mL protein), combined with an equal volume of 1% digitonin on ice for 15 minutes. The pellet was washed twice in IB, re-suspended in 200 μL of IB and 2 μL of 10% Lubrol PX (C12E9; Calbiochem, San Diego, Calif.) on ice for 15 minutes, then layered onto IB and centrifuged at 39,000 rpm for 1 hour, followed by an IB wash. Final protein concentration was ˜1-4 mg/mL of protein by BCA (Pierce).

F1 Removal from SMV's.

F1 subunits were removed from SMVs by adapting previously-established methods. 60 mg SMV's/1 mL IB was treated with 1 mL of 6 mM urea for 5 min. on ice, then centrifuged at 21000×g for 10 min. The pellet was washed 3× in IB (centrifugation at 21000×g for 10 min) and stored in IB.

¹⁴C-Dexpramipexole Binding to SMVs.

Urea-treated or control SMVs were incubated in ¹⁴C-dexpramipexole (GE Healthcare UK, 56 mCi/mmol) overnight (4° C.) in an agitator, then applied to a Centricon Centrifugal Filter Unit with Ultracel YM-10 membrane (Millipore, USA) and centrifuged at 4000×g for 1 hr. SMVs were washed twice with IB. Filter units were incubated in Ultima Gold scintillation liquid (Perkin Elmer Health Sciences, Inc.) overnight. Samples were counted for ¹⁴C with a Beckman Coulter LS 5000TD scintillation counter. In a preliminary experiment, competition with 20 μM unlabeled dexpramipexole produced a 34% reduction in ¹⁴C-dexpramipexole binding to non-urea treated SMVs.

¹⁴C-Dexpramipexole Interaction with Heterologously-Expressed F1F_{O} ATP Synthase Subunits.

The human ORF constructs for alpha, beta, b, c, delta, d, epsilon, gamma and OSCP ATP synthase subunits, tagged with Myc and DDK (Flag) tags were from Origene Technologies (Rockville, Md.). 293T cells were transfected with the above constructs, using the calcium phosphate method (Li et al. 2008). On day-3 post transfection, the cells were lysed and the fusion proteins were bound to the EZview™ Red ANTI-FLAG® M2 Affinity Gel (Sigma, USA), according to the manufacturer's protocol. The proteins were eluted from a portion of the samples and presence of the proteins on the beads was verified by immunoblot analysis, using the mouse anti-Myc antibodyies (Cell signaling Technology). The protein-bound beads were incubated in presence of ₁₄C dexpramipexole overnight at 4° C. in an end-over-end agitator. They were spun at 3000×g in 0.45 um Spin-X centrifugal devices (Corning Life Sciences, USA) for 10 min. The samples were washed three times with PBS and the filter units were incubated in Ultima Gold scintillation liquid (Perkin Elmer Health Sciences, Inc.) overnight. Samples were counted for ¹⁴C, using a Beckman Coulter LS 5000TD scintillation counter.

Oxygen Flux Measurements.

Oxygen uptake was measured in single hippocampal neurons in culture (DIV 14-17). A 2-4 μM (tip diameter) oxygen-sensing electrode oscillated 10 μm close-to and away-from the cell every 3 sec. The difference in current detected at the two positions was translated into oxygen flux. Dexpramipexole (10 μM) or control solution was added after a 5 min. baseline measurement, and flux was measured for >5 min. post-treatment.

Measurements of ATP Levels in Cultured Hippocampal Neurons.

Cultured hippocampal neurons were prepared and plated on 96 well plates. At DIV 14, after exposure to dexpramipexole or vehicle for 12 hours, ATP was measured by a luciferase assay as described herein.

Electrophysiological Recording from Rat Brain-Derived Mitochondria.

Patch-clamp recordings were made from de-energized mitochondria in intracellular solution (120 mM potassium chloride, 8 mM NaCl, 0.5 mM EGTA, 10 mM HEPES, pH adjusted to 7.3) at room temperature (22-25° C.). Pipettes (80-100M0) were filled with the same solution; recordings were made using a Heka 8 amplifier with Vₘ held at positive voltages up to +180 mV. Data were recorded at 20 kHz and filtered at 500-1000 Hz. Dexpramipexole (Knopp Neurosciences, Pittsburgh, Pa.) was prepared as a 10 mM aqueous stock and diluted in intracellular recording solution; cyclosporine A (CSA; Sigma, St. Louis, Mo.) was prepared as an 8 mM stock solution in EtOH and diluted in buffer. Reagents were rapidly perfused into the recording chamber. Peak membrane conductance was measured as the peak amount of current (pA) from zero, converted to pS by assuming a linear I-V relationship. Discrete channel conductances were sorted by activity (% occurrence of a conductance level per unit time measured using pCLAMP 10 software, Molecular Devices, Sunnyvale, Calif.); levels defined as closed (no current), small (<200 pS), intermediate (>200 pS and <750 pS) and large (>750 pS). Mean open time (NPo; open time of discrete single channels times the apparent number of single channels in the patch) was used to assess drug effect

Patch Clamp Recordings from SMVs.

Giga-ohm seals were formed on SMVs using pipettes and solutions as above. Peak membrane conductance was measured as described above.

Western Blot Analysis.

Western blot analysis was performed using standard protocols. Primary antibodies were mouse anti β-subunit 1:1000 (Mitosciences, Eugene Oreg.) and mouse anti-ANT 1:1000 (Santa Cruz Biotechnology, Santa Cruz Calif.).

Measurement of ATP Hydrolysis Using a Luciferin-Luciferase Assay.

ATP hydrolysis in SMVs was measured with the BioVision Aposensor ATP Assay Kit in a 96-well plate with a plate reader (VICTOR3Multilabel Perkin Elmer). SMVs were suspended in IB plus BSA (0.03 mg/mL), ATP solution (final 0.5 mM) and 1 μL of reconstituted ATP-monitoring enzyme. To initiate, 100 μL of Nucleotide Releasing Agent containing Triton X was added and luminescence measured and displayed as percent change in luminescence over time. 3 wells were used for each condition, repeated at least 3× on different SMV isolations.

Measurement of ATP Hydrolysis Using an NADH Assay.

ATP hydrolysis was also measured using an NADH-ATP-synthase kit (Mitosciences, USA; catalog #MS541), according to the manufacturer's protocol with modifications. SMVs were added 20 min. prior to addition of the reagent mix. The rate of change in fluorescence over time as NADH was oxidized was measured as a decrease in absorbance at 340 nm.

Cell Culture Preparation and Maintenance.

Primary cultures of cortical neurons were prepared as described previously, plated at 8×10⁵ cells/well for Seahorse XF24 experiments and grown in Neurobasal A supplemented with B27, 1 mM glutamine and penicillin/streptomycin for 4 days in vitro (DIV). On DIV 7, media without antibiotics was substituted. The cultures were used on DIV 9-10.

Undifferentiated SH-SY5Y human neuroblastoma cells (ATCC, Manasses, Va.) were maintained in humidified 5% CO₂ in 1:1 Ham's F12 nutrient mixture with Glutamax® and Minimal Essential Medium with L-glutamine (MEM-Alpha), 10% FBS, and 1% penicillin/streptomycin (Gibco® Invitrogen Corp., Carlsbad, Calif.).

The C2C12 mouse myoblast cell line (ATCC, Manasses, Va.) was cultured in Dulbecco's Modified Eagle Medium (DMEM; Gibco®; Carlsbad, Calif.) supplemented with 10% fetal bovine serum (FBS), 2 mM GlutaMax® and penicillin/streptomycin. Cells were maintained in 10% CO₂ humidified at 37° C.

Cell Viability and Cellular ATP Quantitation.

Cellular viability was measured using CellTiter-Blue® (Promega Corp., Madison, Wis.) in black-walled optical imaging multiwell plates, and cellular ATP was measured using the CellTiter-Glo® (Promega Corp., Madison, Wis.) luminescence assay in opaque-walled multiwell plates according to the manufacturer's protocol.

Kinetic ATP Synthesis Assay.

Synthesis of ATP in liver mitochondria was measured using an ATP determination kit (A-22066; Molecular Probes, Eugene, Oreg.), supplemented with 1 mM sodium succinate and 500 nM rotenone. Assays were performed in 96-well microplates (Becton Dickinson, Franklin Lakes, N.J.) in a final volume of 110 μL consisting of 10 μL mitochondria (0.1 mg protein/ml final concentration), 10 μL of drug or control buffer and 90 μL ATP-determination reaction mixture. After a 5 min. recording of basal luciferase-generated luminescence, ADP (final concentration, 10 μM, 10 μL) was injected into each well. Luminescence was recorded every 6 sec for 15-20 min.

Cell Culture Oxygen Consumption Experiments.

OCR and ECAR were measured with a Seahorse® XF24 Flux Analyzer (Seahorse Bioscience, Billerica, Mass.). For PSI experiments on C2C12 cells, basal values were assessed in non-buffered DMEM Assay Medium without glucose, glutamine or pyruvate, and supplemented with 25 mM glucose, 6 mM Glutamax®, and 1 mM sodium pyruvate. For primary cortical neurons the assay had 2 modifications: 1) MAS 1 buffer (70 mM sucrose, 220 mM mannitol, 5 mM KH₂PO₄, 5 mM MgCl₂, 2 mM HEPES, 1 mM EGTA, 0.2% FA-free BSA; pH 7.2; Sigma-Aldrich, St. Louis, Mo.) was used instead of Assay Media, and 2) 10× concentrated digitonin (10 μg/mL), rotenone (100 nM) and dexpramipexole (30 μM) or control (equivalent volume of water) were added to the cells 45 minutes prior to assay. Succinate and ADP stocks were diluted to 10× concentrations in pre-warmed XF MAS buffer, pH 7.2. Using a Plate Prep Station (Seahorse Bioscience, Billerica, Mass.), cells were washed with 1 mL buffer and were brought to 607 μL with buffer. Dexpramipexole or water (68 μL; 10× concentrated) was added to the wells, and cells incubated at 37° C. in a non-CO₂ incubator for 45 minutes to 1 hour. Raw basal OCR and ECAR values were normalized as a percent of the mean control value for each individual experiment. Normalized data from experiments were expressed as mean±SEM.

Statistical Analyses and Curve Fitting.

For comparisons involving 2 groups, paired or unpaired Student's t-tests (2-tailed) were used. In all figures, *=p<0.05, **=p<0.01, and ***=p<0.001 to denote significance level, and exact p values are provided in the figure legends. For more than 2 groups, one-way or 2-way analyses of variance (ANOVA), or 2-factor MANOVA, were performed; in the case of a significant F-test, the p value is provided in the figure legend, and pre-planned post hoc comparisons (Bonferroni-corrected t-tests or Tukey's HSD) were performed and significance levels displayed in figures and exact p values provided as described above. Where possible, p values for tests are presented to 2 significant digits. All statistical analyses were performed using GraphPad Prism 5, InStat (GraphPad Software, La Jolla, Calif.) or SPSS (IBM Corporation, Somers, N.Y.).

Other methods that can be used are also described in McNaught, K. S., Perl, D. P., Brownell, A. L., & Olanow, C. W. Systemic exposure to proteasome inhibitors causes a progressive model of Parkinson's disease. Ann Neurol 56, 149-162 (2004); Chan, T. L., Greenawalt, J. W., & Pedersen, P. L. Biochemical and ultrastructural properties of a mitochondrial inner membrane fraction deficient in outer membrane and matrix activities. J. Cell Biol. 45, 291-305 (1970); Pedersen, P. L., Hullihen, J., & Wehrle, J. P. Proton adenosine triphosphatase complex of rat liver. The effect of trypsin on the F1 and F0 moieties of the enzyme. J. Biol. Chem. 256, 1362-1369 (1981). Li, H. et al. Bcl-xL induces Drp1-dependent synapse formation in cultured hippocampal neurons. Proc Natl Acad Sci USA 105, 2169-2174 (2008); Land, S. C., Porterfield, D. M., Sanger, R. H., & Smith, P. J. The self-referencing oxygen-selective microelectrode: detection of transmembrane oxygen flux from single cells. Journal of Experimental Biology 202, 211-218 (1999); Lotscher, H. R., deJong, C., & Capaldi, R. A. Interconversion of high and low adenosinetriphosphatase activity forms of Escherichia coli F1 by the detergent lauryldimethylamine oxide. Biochemistry 23, 4140-4143 (1984); Wang, S. et al. Cellular NAD replenishment confers marked neuroprotection against ischemic cell death: role of enhanced DNA repair. Stroke 39, 2587-2595 (2008), each of which is hereby incorporated by reference in its entirety.

Although the present invention has been described in considerable detail with reference to certain embodiments thereof, other versions are possible. Therefore the spirit and scope of the appended claims should not be limited to the description and the embodiments contained within this specification.

1. A method of identifying a compound that increases oxygen utilization efficiency comprising
contacting a compound that binds a mitochondrial ATP synthase complex with a mitochondria or a sub-mitochondrial vesicle; and
measuring oxygen utilization efficiency,
wherein when the measured oxygen utilization efficiency in the mitochondria or a sub-mitochondrial vesicle increases in the presence of the compound that binds the mitochondrial ATP synthase complex indicates that the compound is a compound that increases oxygen utilization efficiency.

2. The method of claim 1, further comprising identifying a compound that binds to the mitochondrial ATP synthase complex comprising:
contacting a test compound with the mitochondrial ATP synthase complex;
and identifying the test compound as the compound that binds the mitochondrial ATP synthase complex.

3. The method of claim 1, wherein the mitochondria are isolated mitochondria or the mitochondrial ATP synthase is an isolated mitochondrial ATP synthase complex.

4. (canceled)

5. The method of claim 1, wherein a cell comprises the mitochondria and/or the mitochondrial ATP synthase complex.

6-12. (canceled)

13. The method of claim 1 further comprising heterologously expressing and isolating the mitochondrial ATP synthase complex prior to contacting the compound with the mitochondrial ATP synthase complex.

14. The method of claim 1, further comprising measuring ATP synthesis in the presence of the compound, wherein when the measured ATP synthesis is at least maintained in the presence of the compound indicates that the compound increases oxygen utilization efficiency and at least maintains ATP synthesis.

15. The method of claim 1, wherein said mitochondrial ATP synthase complex comprises an F1 head, a b subunit, an OSCP subunit, or a combination thereof.

16. The method of claim 1, wherein said compound binds to an F1 head, a b subunit, an OSCP subunit, or a combination thereof.

17. The method of claim 1, wherein said measuring oxygen utilization comprises comparing the compound to a positive or a negative control.

18. (canceled)

19. The method of claim 2, wherein said identifying the test compound as an identified test compound that binds the mitochondrial ATP synthase complex comprises determining whether said test compound can competitively inhibit the binding of dexpramipexole to the mitochondrial ATP synthase complex.

20. The method of claim 17, wherein said positive control comprises a detectable label.

21-24. (canceled)

25. The method of claim 1, wherein measuring oxygen utilization efficiency comprises measuring inhibition of mitochondrial conductance.

26-29. (canceled)

30. The method of claim 1, further comprising identifying a compound as a compound that treats a neurodegenerative disease comprising contacting a subject with a neurodegenerative disease with the compound identified as a compound that that increases oxygen utilization efficiency,
wherein a compound that improves the condition of the subject inhibits the progression of the neurodegenerative disease, ameliorates the neurodegenerative disease indicates the compound as a compound that treats a neurodegenerative disease.

31. The method of claim 1, further comprising identifying a compound as a compound that is a neuroprotectant comprising contacting a subject with a neurodegenerative disease with the compound identified as a compound that that increases oxygen utilization efficiency,
wherein a compound that improves the condition of the subject inhibits the progression of the neurodegenerative disease, ameliorates the neurodegenerative disease indicates the compound as a compound that treats a neurodegenerative disease.

32. A method of identifying a compound that increases oxygen utilization efficiency and at least maintains ATP synthesis comprising:
contacting a mitochondria or a sub-mitochondrial vesicle with a compound that binds to a mitochondrial ATP synthase complex and measuring oxygen utilization efficiency and ATP synthesis,
wherein a compound that increases oxygen utilization efficiency and enhances or increases ATP synthesis identifies the compound as a compound that increases oxygen utilization efficiency and at least maintains ATP synthesis.

33. The method of claim 32, further comprising determining whether said compound can inhibit the binding of dexpramipexole to the mitochondrial ATP synthase complex, wherein a compound that inhibits the binding of dexpramipexole to the mitochondrial ATP synthase complex identifies the compound as a compound that binds to the mitochondrial synthase complex.

34. The method of claim 32, further comprising comparing oxygen utilization efficiency and ATP synthesis in the presence of the compound to oxygen utilization efficiency and ATP synthesis in the presence of dexpramipexole, wherein a compound that at least maintains oxygen utilization efficiency and at least maintains ATP synthesis as compared to dexpramipexole identifies the test compound as a compound that increases oxygen utilization efficiency and at least maintains ATP synthesis.

35-47. (canceled)

48. The method of claim 32, comprising heterologously expressing and isolating the mitochondrial ATP synthase complex prior to contacting the compound with the mitochondrial ATP synthase complex.

49-53. (canceled)

54. The method of claim 32, further comprising identifying a compound as a compound that treats a neurodegenerative disease comprising contacting a subject with a neurodegenerative disease with the compound identified as a compound that that at least increases oxygen utilization efficiency and at least maintains ATP synthesis,
wherein a compound that improves the condition of the subject, inhibits the progression of the neurodegenerative disease, or ameliorates the neurodegenerative disease indicates the compound as a compound that treats a neurodegenerative disease.

55. A method of identifying a compound that treats a neurodegenerative disease comprising:
contacting a subject with a neurodegenerative disease with a test compound, wherein said test compound is a compound that increases oxygen utilization efficiency, a compound that at least maintains ATP synthesis, and/or a compound that binds to a mitochondrial ATP synthase complex,
wherein a compound that improves the condition of the subject, inhibits the progression of the neurodegenerative disease, or ameliorates the neurodegenerative disease indicates the compound as a compound that treats a neurodegenerative disease.

56. The method of claim 55, further comprising identifying a compound that increases oxygen utilization efficiency, a compound that at least maintains ATP synthesis, and/or a compound that binds to a mitochondrial ATP synthase complex.

57. The method of claim 55, wherein said test compound is a compound that increases oxygen utilization efficiency, at least maintains ATP synthesis, and binds to a mitochondrial ATP synthase complex.

58-68. (canceled)
